(19) United States
(12) Patent Application Publication
(10) Pub. No.: US 2025/0223539 A1
(43) Pub. Date: Jul. 10, 2025

(54) METHODS AND SYSTEMS FOR ENHANCING METHANOGENESIS FOR SUBSURFACE METHANE PRODUCTION OPTIMIZATION

(71) Applicant: SAUDI ARABIAN OIL COMPANY, Dhahran (SA)

(72) Inventors: Klemens Katterbauer, Dhahran (SA); Abdallah A. AlShehri, Dhahran (SA); Abdulaziz S. Al Qasim, Dammam (SA); Ali Abdullah AlYousif, Dhahran (SA)

(73) Assignee: SAUDI ARABIAN OIL COMPANY, Dhahran (SA)

(21) Appl. No.: 18/404,455

(22) Filed: Jan. 4, 2024

(51) Int. Cl. C12M 1/36; C12M 1/107; C25B 1/04

(52) U.S. Cl. CPC C12M 41/48; C12M 21/04; C25B 1/04

(57) ABSTRACT

A method for optimal production of methane from a storage horizon configured as an underground bioreactor, the method including obtaining environmental data for a renewable energy facility that produces hydrogen and obtaining process data from an industrial facility that produces carbon dioxide. The method further includes injecting the produced hydrogen, the produced carbon dioxide, and a selection of microbes, the selection defined by a set of microbe parameters, into the bioreactor. The bioreactor produces a quantity of methane that is controlled by, at least in part, a set of operation parameters. The method further includes determining, with a composite artificial intelligence model, a predicted methane production from the bioreactor based on the environmental data, the process data, the set of microbe parameters, and the set of operation parameters and adjusting, automatically, the set of operation parameters and the set of microbe parameters to optimize methane production.

### BACKGROUND

Developing new forms of sustainable energy, effectively managing existing sources of sustainable energy including the storage of sustainably-generated energy without on-demand availability, and properly managing sources of pollution are each important objectives. For example, storing electrical energy produced by renewable energy facilities is a challenge. A sustainable option is the conversion of electrical energy into chemical energy that can be more easily stored. One possibility in this regard includes the conversion of electrical energy into hydrogen via electrolysis and transporting the hydrogen to and storing the hydrogen in a storage horizon (i.e., underground gas storage) for extraction and use at a later period (e.g., corresponding with an increase in energy demands).

Additionally, many industrial facilities output carbon dioxide as a byproduct of their operations. Further, certain types of microbes, broadly referred to as methanogens, are capable of synthesizing methane from carbon dioxide via a metabolic reaction using hydrogen as a reducing agent. As such, an underground bioreactor can be formed, in principle, by injecting hydrogen and carbon dioxide into a storage horizon supportive of microbes for methane synthesis. Optimal methane production from such an underground bioreactor is highly dependent on factors relating to the geological environment and the above-ground installations responsible for the production of hydrogen and carbon dioxide. These factors may be interdependent and have interactions that dramatically affect, at least, methane production and thus the overall success of the underground bioreactor. Accordingly, there exists a need to relate observable factors to methane production and intelligently adjust controllable parameters governing the underground bioreactor and associated above-ground installation to optimally operate such a system.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

Embodiments disclosed herein generally relate to a method for optimal production of methane from a storage horizon configured as an underground bioreactor, the method including obtaining environmental data for a renewable energy facility that produces hydrogen and obtaining process data from an industrial facility that produces carbon dioxide. The method further includes injecting, at least some of the hydrogen produced by the renewable energy facility, at least some of the carbon dioxide produced by the industrial facility, and a selection of microbes from a microbe population into the underground bioreactor. The selection of microbes is defined by a set of microbe parameters. The underground bioreactor produces a quantity of methane, and the quantity of methane produced is controlled by, at least in part, a set of operation parameters. The method further includes determining, with a composite artificial intelligence AI model including a first artificial intelligence model, a predicted methane production from the underground bioreactor based on the environmental data, the process data, the set of microbe parameters, and the set of operation parameters. The method further includes determining, with an optimizer applied to the composite AI model, an optimal set of operation parameters and optimal set of microbe parameters such that the predicted methane production is optimized and adjusting, automatically, the set of operation parameters and the set of microbe parameters to the optimal set of operation parameters and the optimal set of microbe parameters, respectively.

Embodiments disclosed herein generally relate to a system including a renewable energy facility that produces hydrogen and an industrial facility that produces carbon dioxide, where the operation of the renewable energy facility and the industrial facility is defined by a set of operation parameters. The system further includes a plurality of facility devices disposed throughout the renewable energy facility and the industrial facility, the plurality of facility devices gathering operation data from the renewable energy facility and the industrial facility. The system further includes a microbe population providing a selection of microbes, where properties of the selection of microbes are defined by a set of microbe parameters. The system further includes a storage horizon configured as an underground bioreactor that produces methane, where the quantity of produced methane is controlled, at least in part, by a set of injection materials. The injection materials include the hydrogen from the renewable energy facility, the carbon dioxide from the industrial facility, and the selection of microbes from the microbe population. The system further includes a control system configured to adjust one or more facility devices in the plurality of facility and a computer. The computer is configured to obtain the operation data for the renewable energy facility and the industrial facility, obtain the set of operation parameters for the renewable energy facility and the industrial facility, obtain the set of microbe parameters for the selection of microbes, and determine, with a composite AI model including a first AI model, a predicted methane production based on the operation data, the set of operation parameters, and the set of microbe parameters. The computer is further configured to determine, with an optimizer applied to the composite AI model, an optimal set of operation parameters and optimal set of microbe parameters such that the predicted methane production is optimized, and adjust, automatically, the set of operation parameters and the set of microbe parameters to the optimal set of operation parameters and the optimal set of microbe parameters, respectively, to optimize the predicted methane production.

Embodiments disclosed herein generally relate to a non-transitory computer-readable memory including computer-executable instructions stored thereon that, when executed on a processor, cause the processor to perform the following steps. The steps include obtaining environmental data for a renewable energy facility that produces hydrogen and obtaining process data from an industrial facility that produces carbon dioxide. The steps further include determining, with a composite artificial intelligence (AI) model, a predicted methane production from an underground bioreactor based on the environmental data, the process data, and a set of microbe parameters in view of a set of operation parameters that control the operation of the renewable energy facility and the industrial facility. The steps further include determining, with an optimizer applied to the composite AI model, an optimal set of operation parameters and optimal set of microbe parameters such that the predicted methane production is optimized and adjusting, automatically, the set of operation parameters and the set of microbe parameters to the optimal set of operation parameters and the optimal set of microbe parameters, respectively.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn are not necessarily intended to convey any information regarding the actual shape of the particular elements and have been solely selected for ease of recognition in the drawing.

FIG. 1 depicts an underground bioreactor environment in accordance with one or more embodiments.

FIG. 2 depicts a system in accordance with one or more embodiments.

FIG. 3 depicts a system in accordance with one or more embodiments.

FIG. 4 depicts a system in accordance with one or more embodiments.

FIG. 5 depicts a system in accordance with one or more embodiments.

FIG. 6 depicts a flowchart in accordance with one or more embodiments.

FIG. 7 depicts a flowchart in accordance with one or more embodiments.

FIG. 8 depicts a neural network in accordance with one or more embodiments.

FIG. 9A depicts a recurrent neural network in accordance with one or more embodiments.

FIG. 9B depicts an unrolled recurrent neural network in accordance with one or more embodiments.

FIG. 9C depicts a long short-term memory network in accordance with one or more embodiments.

FIG. 10 depicts a system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. For example, a “renewable energy facility,” may include any number of “renewable energy facilities,” without limitation.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

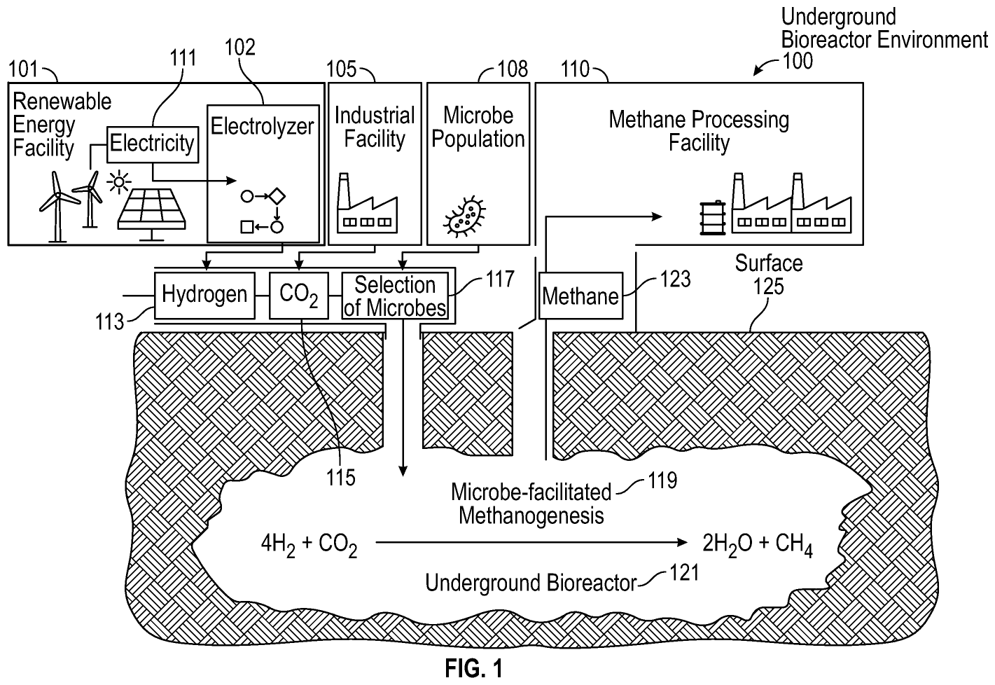

In the following description of FIGS. 1-10, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

In general, embodiments of the disclosure include systems and methods for optimizing methane production from a storage horizon configured as a bioreactor. Herein, a “storage horizon,” is defined as a geological underground formation, group of formations, or part of a formation that receives and/or stores injected fluids. The injected fluids may be multiphase and be composed of a variety of solid, liquid, and gaseous constituents. In one or more embodiments, a storage horizon may be a depleted natural gas or oil field, aquifer, or salt formation. A “bioreactor,” is a structure in which injected substances are converted and/or combined to create output substances through one or more biological and/or chemical reactions.

In accordance with one or more embodiments, systems of the instant disclosure include a renewable energy facility that produces hydrogen, an industrial facility that produces carbon dioxide (likely, as a byproduct of another process such as gas refinement), and an underground bioreactor (“bioreactor”). A “renewable energy facility,” is any facility that generates energy from renewable resources, for example, a solar farm wherein energy from the sun in the form of radiation is converted to electrical energy using solar panels. In one or more embodiments, the renewable energy facility, the industrial facility, and the bioreactor are in proximity to one another, subject to physical and environmental constraints. For example, an above-surface environment of a storage horizon may not be suitable for a renewable energy facility (e.g., on average low winds and low solar intensity). In one or more embodiments, methane is produced in a storage horizon configured as a bioreactor. The substances injected into the bioreactor include hydrogen sourced from a renewable energy facility, carbon dioxide (CO₂) sourced from an industrial facility, and a selection of microbes sourced from a microbe population. An “industrial facility,” in this case may be any facility that produces CO₂ directly and/or as a byproduct of its operations. The microbe population may include any of the methanogenic archaea capable of synthesizing methane from CO₂. The selection of microbes injected is controlled through assignation of a set of microbe parameters to a given state or value. The efficiency of methane production and overall quantity of methane acquired are affected by both the set of microbe parameters and a set of operation parameters that define a bioreactor optimization system and its operation. In general, an interaction exists between the set of microbe parameters, the set of operation parameters, and the production rate and quantity of methane produced from the underground bioreactor. In one or more embodiments, the set of microbe parameters and the set of operation parameters are adjusted, automatically and in real time, to maximize the production of methane in the bioreactor.

In one or more embodiments, methane production in the bioreactor is predicted using a composite artificial intelligence (AI) model, including at least one AI model, based on (or accepts as inputs), at least, the set of microbe parameters and the set of operation parameters. In one or more embodiments, the AI model may further make use of environmental data from the renewable energy facility (e.g., the total elapsed time of sunlight upon solar panels at a solar farm) that affect the production of hydrogen and operation data from the industrial facility (e.g., the current rate of fossil fuel burning) that affect the production of CO₂. Using the composite AI model to predict methane production, both an optimal set of operation parameters and optimal set of microbe parameters may be determined by applying an optimizer to the composite AI model.

FIG. 1 shows a schematic diagram in accordance with one or more embodiments. More specifically, FIG. 1 illustrates an underground bioreactor environment (100) that includes an underground bioreactor (“bioreactor”) (121) located beneath the Earth's surface (“surface”) (125). In proximity to the bioreactor (121) is a renewable energy facility (101), an industrial facility (105), a microbe population (108), and a methane processing facility (110). In one or more embodiments, the microbe population (108) may not be a physical population but rather represents an ability to select and/or source microbes. The underground bioreactor (121) may be configured in any storage horizon suitable for gas storage and extraction. The renewable energy facility (101), industrial facility (105), and microbe population (108) are each able to inject material into the bioreactor (121) owing to their mutual proximity. In one or more embodiments, hydrogen (113) sourced from the renewable energy facility (101), CO₂ (115) sourced from the industrial facility (105), and a selection of microbes (117) sourced from the microbe population (108) may each be injected into the underground bioreactor (121). Inside the bioreactor (121), microbe-facilitated methanogenesis (119) takes place utilizing the injected hydrogen (113), CO₂ (115), and selection of microbes (117). The resultant methane may be collected and further processed by a methane processing facility (110).

In one or more embodiments, a renewable energy facility includes a means to store energy as hydrogen, the energy originally being of a variety of forms, including electrical and chemical. For example, hydrogen may be produced by a renewable energy facility (101) that generates electricity (111), where the electricity (111) is used to separate hydrogen from H₂O via electrolysis inside of an electrolyzer (102). The term “electrolysis” refers to a technique that uses direct electric current to drive an otherwise non-spontaneous chemical reaction. For example, the electrolysis of water is the process of using electricity to electrochemically decompose water into oxygen and hydrogen. The term “electrolyzer” refers to a unit where this chemical reaction may take place.

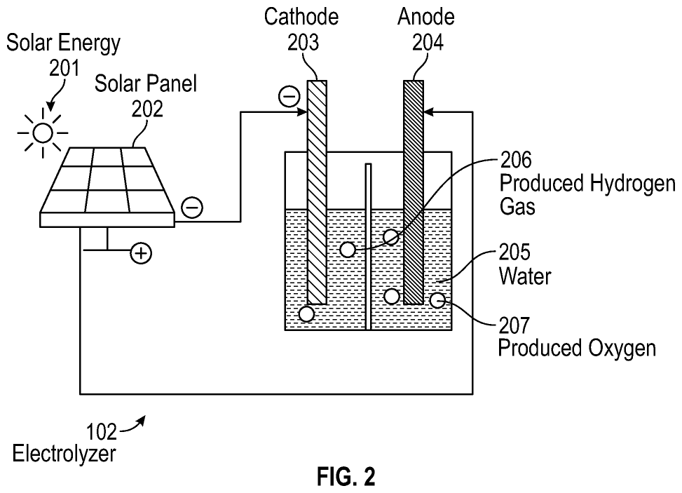

FIG. 2 illustrates an electrolysis technique for producing hydrogen gas from water. Hydrogen gas may be produced from water by using differing configurations, materials, and method steps as known to those skilled in the art. Thus, the depiction of FIG. 2 is provided solely as an example and should not be considered limiting. As shown in FIG. 2, an electrolyzer (102), including a cathode (203) and an anode (204), is in electrical communication with a solar panel (202) that converts solar energy (201) into electricity. The source of electricity need not be a solar panel. For example, a wind turbine that uses wind to generate electricity may be used instead of a solar panel, or the source of electricity may be a geothermal energy system. When an electric current is passed through water (205), water (205) is electrochemically decomposed into hydrogen gas (206) and oxygen (207). This hydrogen gas (206) produced may be collected for injection in an underground bioreactor.

Returning to FIG. 1, in one or more embodiments, the storage horizon configured as an underground bioreactor (121) is provided with reactants for producing methane, including hydrogen (113), CO₂ (115), and a selection of microbes (117). Inside the bioreactor (121), microbe-facilitated methanogenesis (119) takes place according to the reaction 4H₂+CO₂→2H₂O+CH₄. Generally, complex interactions between the hydrogen (113), CO₂ (115) and selection of microbes (117) exist such that configuring the bioreactor for optimal methane production is a difficult and laborious task. Further, the state and behavior of bioreactor may be transient according to the availability of constituents (i.e., hydrogen, CO₂, microbes) for methane production which may or may not vary (e.g., hydrogen production rates may correspond to environmental factors such as wind and solar intensity).

In some embodiments, fluid from the bioreactor (121) may be extracted. For example, the methane (123) produced in the bioreactor may be extracted for other purposes, including further processing or transported elsewhere for use and/or dissemination. In one or more embodiments the methane (123) may be transported to a methane processing facility (110).

Regarding the components of the underground bioreactor environment, the various facilities may have associated with them both data, that describe the status of the given facility, and parameters, that define and control one or more aspects of their operation. For example, the renewable energy facility may have associated “environmental data,” that describes environmental factors related to the production of renewable energy, as well as “renewable energy facility parameters,” that define the operation of the renewable energy facility. Likewise, the industrial facility may have associated “process data” that describes characteristics of the processes taking place in the industrial facility related to the production of CO₂, as well as “industrial facility parameters,” that define the operation of the industrial facility. Lastly, the selection of microbes from the microbe population may also have an associated set of “microbe parameters,” that define features of the selection of microbes that are injected into the bioreactor. In one or more embodiments, the set of microbe parameters control the type (where more than one class or strain may be used) and quantity of microbes present in the bioreactor, where the type and quantity of microbes in the bioreactor may be altered, for example, via injection of a selection of microbes into the bioreactor. Further, in controlling the type and quantity of microbes in the bioreactor, a temporal history of the type and quantity of microbes in the bioreactor, as well as environmental conditions (and a history of those conditions) in the bioreactor such as temperature and pressure, may be considered.

In one aspect, embodiments disclosed herein relate to a system for determining the set of operation parameters, including the renewable energy facility parameters and industrial facility parameters, and the set of microbe parameters that optimize methane production from a storage horizon configured as a bioreactor. The optimal set of operation parameters and the optimal set of microbe parameters are determined with a composite artificial intelligence (AI) model, including at least one AI model, taking into consideration the current state of the renewable energy facility producing hydrogen via the environmental data and the renewable energy facility parameters, the current state of the industrial facility via the process data and industrial facility parameters, and the properties of the selection of microbes via the set of microbe parameters. In accordance with one or more embodiments, the renewable energy facility parameters, the industrial facility parameters, and the set of microbe parameters may be adjusted automatically, and in real-time, through control systems disposed throughout the various facilities (i.e., control system of the renewable energy facility, control system of the industrial facility, etc.) capable of receiving commands, the commands based on the composite AI model under optimization.

In accordance with one or more embodiments, operation data (including, in some embodiments, environmental data for a renewable energy facility and process data from an industrial facility) are processed with a composite artificial intelligence (AI) model to predict a production (rate and/or quantity) of methane from a bioreactor. In turn, the predicted methane production is used to determine an optimal set of operation parameters (including, in some embodiments, both the renewable energy facility parameters and industrial facility parameters), and an optimal set of microbe parameters for the production of methane via microbe-facilitated methanogenesis inside of a storage horizon configured as a bioreactor. Artificial intelligence, broadly defined, is the extraction of patterns and insights from data. The phrases “artificial intelligence”, “machine learning”, “deep learning”, and “pattern recognition” are often convoluted, interchanged, and used synonymously throughout the literature. This ambiguity arises because the field of “extracting patterns and insights from data” was developed simultaneously and disjointedly among a number of classical arts like mathematics, statistics, and computer science. For consistency, the term artificial intelligence (AI), will be adopted herein, however, one skilled in the art will recognize that the concepts and methods detailed hereafter are not limited by this choice of nomenclature.

Artificial intelligence (AI) model types may include, but are not limited to, neural networks, random forests, generalized linear models, and Bayesian regression. AI model types are usually associated with additional “hyperparameters” which further describe the model. For example, hyperparameters providing further detail about a neural network may include, but are not limited to, the number of layers in the neural network, choice of activation functions, inclusion of batch normalization layers, and regularization strength. The selection of hyperparameters surrounding a model is referred to as selecting the model “architecture.” Generally, multiple model types and associated hyperparameters are tested and the model type and hyperparameters that yield the greatest predictive performance on a hold-out set of data is selected.

As noted, the objective of the composite AI model, composed of at least one AI model, is to determine the production of methane from an underground bioreactor in view of environmental data from an associated renewable energy facility (e.g., the total elapsed time of sunlight upon solar panels at a solar farm) that affects the production of hydrogen and operation data from an associated industrial facility. Further, in some embodiments, the composite AI model is further informed by the set of operation parameters and the set of microbe parameters.

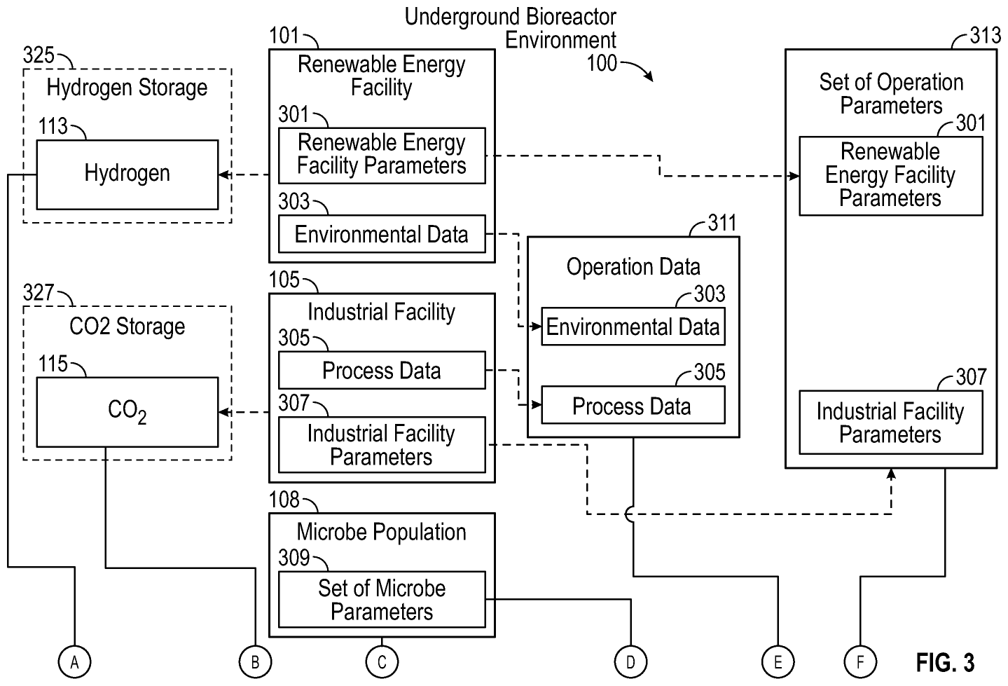

FIG. 3 depicts an instance of the underground bioreactor environment (100) of FIG. 1, meaning that the two structures need not be identical. However, for concision, FIG. 3 does not make use of new numeric labels for previously described, like elements. As such, the underground bioreactor environment (100) includes a renewable energy facility (101), industrial facility (105), and microbe population (108) associated with and capable of injecting substances into an underground bioreactor (121). It is noted that embodiments of the instant disclosure are not limited to underground bioreactors and the facilities that inject material into the underground bioreactor as depicted in FIG. 3. For example, while there may only be one renewable energy facility connected to the underground bioreactor in one embodiment, in other embodiments there may be a plurality renewable energy facility connected to the bioreactor. As seen in FIG. 3, the renewable energy facility (101) has associated with itself environmental data (303) and renewable energy facility parameters (301). Likewise, the industrial facility (105) has associated with itself process data (305) and industrial facility parameters (307). The microbe population (108) has associated with itself a set of microbe parameters (309).

As shown, each of the facilities associated with the underground bioreactor (121) inject one or more substances into the bioreactor to facilitate the production of methane. The renewable energy facility (101) sources hydrogen (113) into the bioreactor, the industrial facility (105) sources carbon dioxide (CO₂) (115) into the bioreactor, and the microbe population (108) sources a selection of microbes (309). The listed injected substances may be multiphase and be composed of a variety of solid, liquid, and gaseous constituents. Collectively, these substances may be referred to as a set of injection substances (324). The set of injection substances are then used for microbe-facilitated methanogenesis, the output of which is water (329) and methane (123).

The status of the renewable energy facility (101) is described, at least in part, by environmental data (303). In one or more embodiments, the environmental data (303) may include, for example, the total elapsed time of sunlight upon solar panels at a solar farm, the ambient temperature, and other weather forecast data (e.g., barometric pressure). Alternatively, the environmental data (303) may include the wind speed and direction at a wind farm. In one or more embodiments, the operation of the renewable energy facility (101) is defined by renewable energy facility parameters (301). The renewable energy facility parameters may include defining the orientation of solar panels at a solar farm which affects the generation of electricity, or the arrangement of wind turbines at a wind farm. In some embodiments, the hydrogen (113) sourced by the renewable energy facility (101) may be injected directly into the underground bioreactor (121) once it is acquired. In other embodiments, the hydrogen (113) may first be transported to a location for hydrogen storage (325) rather than be directly injected into the bioreactor (121). The storage systems for the hydrogen (325) may be its own physical structure or it may be a structure of the renewable energy facility (101). In the case of the underground bioreactor (121) used to produce methane, at least some of the hydrogen (113) produced by the renewable energy facility (101) is injected into the bioreactor (121), and the properties of the injected hydrogen (113), such as the quantity of injected hydrogen (113) or the timing of the injection may be included among the renewable energy facility parameters (301).

In one or more embodiments, CO₂ is produced by an industrial facility (105) directly and/or indirectly as a byproduct of its operation. The status of the industrial facility is described, at least in part, by process data (305). For example, the process data (305) may include the total quantity of fossil fuels available to be consumed, and thereby emit CO₂, by the industrial facility (105), the rate of flow of various fluids to and from the industrial facility, the temperature and/or pressure inside different chambers. The process data (305) may further include measurements of other carbon compounds being produced by the industrial facility (105), such other oxocarbons like carbon monoxide and oxocarbon anions like carbonate among other metrics not listed. In one or more embodiments, the operation of the industrial facility (105) is defined by industrial facility parameters (307). The industrial facility parameters may include defining the rate at which fossil fuels are being consumed and thereby emitting CO₂, or defining the temperature and/or pressure inside different chambers, for example. In some embodiments, the CO₂ (115) sourced by the industrial facility (105) may be injected directly into the underground bioreactor (121) once it is acquired. In other embodiments, the CO₂ (115) sourced by the industrial facility (105) may first be transported to a location for CO₂ storage (327). The storage systems for the hydrogen for the CO₂ (327) may be its own physical structure or it may be a structure of the industrial facility (105). In the case of the underground bioreactor (121) used to produce methane, at least some of the CO₂ (115) produced by the industrial facility (105) is injected into the bioreactor (121), and the properties of the injected CO₂ (115), such as the quantity of injected CO₂ (115), or the timing of injection, may be included among the industrial facility parameters.

In one or more embodiments, a selection of microbes is sourced from a microbe population (108), the microbe population (108) including any of the methanogenic archaea capable of synthesizing methane from CO₂, and injected into the bioreactor (121). These specific microbe strains can transform CO₂ through their metabolic reaction into the desired methane gas. The expression, “microbe,” is purposely chosen over the term bacteria because all methane producing microbes belong to the archaea domain. Solely, the methanogenic archaea are capable of synthesizing methane. Based on their biological classification, the relevant microbes may be referred to as methanogens for simplicity. Methanogens are a widespread class, and their living environments range from the subsurface to the intestinal tract of animals, insects or humans. At the time of writing, the methanogens are organized into four different classes with multiple families with 161 individual methanogen strains. The current classes of methanogens are the Methanobacteria, Methanococci, Methanopyri and Methanomicrobia. The largest class is the Methanomicrobia class with 90 individual strains stands in contrast to the single strain Methanopyri class. The second largest class is the Methanobacteria with 55 individual strains. Further, there are 15 individual species of Methanococci.

During the hydrogenotrophic methanation process CO₂ is transformed into methane and water. The reaction is a strong exothermic conversion process. The microbes use the dissolved carbon dioxide as a carbon source and require electrons from another source, such as hydrogen, for their metabolisms. Various methanogens can utilize other electron donors such as formate or acetate to produce methane gas. Some strains are unable to utilize hydrogen as an electron donor and are specialized on other electron donors. A number of methanogens require other growth conditions such as a minimum sodium chloride (NaCl) concentration or the presence of certain vitamins, yeast extract or rumen fluid, which are either already present in fluids of the storage horizon (e.g., ground water) or can be injected and mixed with fluids of the storage horizon.

Methanogen strains possess variable attributes regarding their environmental preferences of pH value, temperature, and salinity, and regarding nutrition. Differences occur between the separate biological classes but are also prevalent between related family strains. Adaptation to the predominant living conditions is a requirement of growth. This is because in an underground environment temperature, salinity, pH-value and nutrition concentration can be extreme, thereby restricting microbial growth. Exceeding the maximum feasible conditions of a strain decelerates the cell creation process and as a result the methane production.

Regarding temperature preferences, the methanogens are spread through all categories. Methanogens live and thrive in a mesophilic, thermophilic and hyperthermophilic and even psychrophilic environment. The optimal temperature is the temperature where potentially the greatest growth rate occurs. The lowest optimal living temperature know for a methanogen is 15 C by the methanomicrobia class strain Methanogenium frigidum. The highest known optimal temperature is 98° C. by the methanopyri class strain Methanopyrus kandleri. Microbes can exceed their optimal temperature towards a maximum growth temperature. The highest maximum growth temperature is 110 C by the Methanopyrus kandleri strain. There are nine strains that prefer a hyperthermophile environment and 14 strains which prefer psychrophilic environment. There are a considerable number of methanogens that favor elevated temperatures above 60 C, which demonstrates the temperature resilience.

The critical NaCl concentrations of some methanogens are not available. Two known strains, which are extremely halophile are the Methanocalculus halotolerans and natrophilus. Further, 16 strains survive under halophilic conditions, which shows that specific methanogens have a considerable salinity tolerance. Whereas the halophilic methanogens are spread over the mesophilic, thermophilic and hyperthermophile temperature range both extremely halophile strains dwell in a mesophilic environment. Demonstrating that elevated resistance against a chosen property does not automatically equate to other property resistances. Contrary to the salinity resistant strains, there are nine fragile strains which endure low amount of salinity. The variety in preferences shows the diversity between the strains and demonstrates that a strain suited for industrial purposes must possess a diversity of attributes.

Concerning the pH-level, methanogens mostly prefer neutral conditions with a small acidic inclination of around pH 6.5 to 7. There are few strains that grow optimally under acidic conditions of pH 5. Three to four strains favor alkaline conditions with an optimal pH value of 9 to 9.5. The critical pH-value ranges for the majority of methanogens from pH 5 to 8.5. There are a few strains that can endure higher alkaline conditions with an critical pH-value of 10 and a selected few strains that can endure acidic conditions of 4. The strains enduring the elevated alkaline conditions are the Methanobrevibacter gottschalkii, millerae, and olleyae, the Methanospirillum hungatei, and stamsii, the Methanosalsum natronophilum and zhilinae. Further, Methanocalculus alkaliphilus and halophilus are the two extremely halophilic methanogen strains. Regarding the extreme acidic range, the Methanobacterium espanolae, and palustre, the Methanocaldococcus bathoardescens, the Methanococcus aeolicus, the Methanothermococcus okinawensis, the Methanoregula boonei, the Methanosarcina baltica and soligelidi are known.

In summary, methanogens require particular living conditions and resources for their sustained longevity and reproduction. Therefore, the selection of microbes (117) may also include additional material intended to foster the growth of the microbes. This additional material may include substances to influence the pH and the salinity of the underground bioreactor (121), yeast extract, rumen fluid, vitamins, or some combination of substances listed as well as those not listed. Hereafter, “selection of microbes” refers to both the sample of injected organisms that synthesize methane from CO₂ along with any additional material injected intended to foster and support their growth inside of the bioreactor (121). The properties of the selection of microbes (117) are defined by a set of microbe parameters (309). The set of microbe parameters (309) may include the quantity of injected microbes, the particular strain or combination of strains of methanogen, the ratio of the quantities belonging to each strain of methanogen in the case where more than one strain is injected, or the quantities and types of various of injected materials intended to foster and support the growth of the microbes. In one or more embodiments, the microbe population is located in proximity to the bioreactor (121) with a means for injecting the selection of microbes (117) that it sources.

Returning to FIG. 3, in one or more embodiments, a bioreactor optimization system (315) interacts with the bioreactor (121), the renewable energy facility (101), industrial facility (105), and microbe population (108). Interaction is facilitated via one or more communication systems. A communication system transmits and receives signals, where the signals may represent data values (e.g., temperature or voltage corresponding to a temperature acquired using a thermocouple) or commands that upon receipt alter the state or behavior of a device (e.g., a valve) or system. In one or more embodiments, the communication systems are connected to one or more sensors measuring and recording data located within renewable energy facility (101), industrial facility (105), microbe population (108), and/or the bioreactor (121). For example, a communication system may be connected to a sensor within the renewable energy facility (101) that records the environmental data (303) such as ambient temperature, wind speed, wind direction, and/or solar intensity. Additionally, in one or more embodiments, the communication system may also be connected to controllers. Controllers can adjust the parameters that define a system or the operation of a system. For example, a communication system may be connected to a controller within the renewable energy facility (101) that can adjust the renewable energy facility parameters (301), for example, issuing a command to adjust the orientation of a solar panel.

As seen, data from the various facilities that output members of the set of injection substances (324) into the bioreactor are collected from a plurality of devices and sensors disposed therein and are collectively stored as operation data (311). From the renewable energy facility this may include the environmental data (303), and from the industrial facility this may include the process data (305). Environmental data (303) may include, for example, the total elapsed time of sunlight upon solar panels at a solar farm, the ambient temperature, and other weather forecast data (e.g., barometric pressure). Alternatively, the environmental data (303) may include the wind speed and direction at a wind farm, for example. Process data (305) may include, for example, the total quantity of fossil fuels available to be consumed, and thereby emit carbon dioxide, by the industrial facility (105), the rate of flow of various fluids to and from the industrial facility, the temperature and/or pressure inside different chambers, among others not listed. As previously discussed, the renewable energy facility (101) may include only one renewable energy facility, or it may include a plurality of renewable energy facilities, each producing hydrogen (113) to be injected into the bioreactor (121). Likewise, the industrial facility (105) may include only one industrial facility, or it may include a plurality of industrial facilities, each producing CO₂ (115) to be injected into the bioreactor (121). Accordingly, the operation data (311) contains all data gathered from the plurality of facilities associated with the underground bioreactor (121). One with ordinary skill in the art will appreciate that additional devices and sensors may be employed among the various facilities associated with the bioreactor and that additional operation data may be collected without departing from the scope of this disclosure.

Considering again the various facilities associated with the bioreactor, including the renewable energy facility (101) and the industrial facility (105), the parameters that define their operation may be collectively referred to as a set of operation parameters (313). Accordingly, the operation parameters (313) define aspects of the operation of every facility associated with the underground bioreactor (121). The set of operation parameters (313) may include the renewable energy facility parameters and the industrial facility parameters. Regarding the renewable energy facility parameters, among those included may be defining the orientation of solar panels at a solar farm which affects the generation of electricity, or the arrangement of wind turbines at a wind farm, or the amount of hydrogen injected to the bioreactor, for example. Regarding the industrial facility parameters, among those included may be defining the rate at which fossil fuels are being consumed and thereby emitting CO₂, or defining the temperature and/or pressure inside different chambers, or defining the amount of CO₂ injected to the bioreactor, for example. The operation parameters (313) may also include defining properties of the injected hydrogen relative to the injected CO₂, such as a compression ratio, ratio by weight, or a relative timing of the respective injections. Similarly, the set of microbe parameters (309) associated with the microbe population (108) define the qualities of the selection of microbes (117) injected into the bioreactor (121). The set microbe of parameters (309) may include the quantity of injected microbes, the particular strain or combination of strains of methanogen, the ratio of the quantities belonging to each strain of methanogen in the case where more than one strain is injected, or the quantities and types of various of injected materials intended to foster and support the growth of the microbes, for example.

In accordance with one or more embodiments, the operation data (311), set of operation parameters (313), and the set of microbe parameters (309) are processed by a composite AI model (317). In one or more embodiments, the result of the composite AI model is a prediction of the methane that will be produced (321) and a prediction of the water that will be produced (319) from the bioreactor given, at least, the operation data (311) in view of or along with the set of operation parameters (313) and the set of microbe parameters (309). The prediction for methane production can be given as a rate, a quantity over a given time period, or both. While FIG. 3 depicts the composite AI model (317) as being applied to an underground bioreactor (121) and associated facilities, herein, the minimum number of renewable energy facilities (101), industrial facilities (105), and bioreactors (121), required to form a functioning bioreactor (121) are each one. As such, in one or more embodiments, the composite AI model (317) is applied to a single renewable energy facility (101), a single industrial facility (105), and a single bioreactor (121). In one or more embodiments, the composite AI model (317) is applied to a single renewable energy facility (101), a single industrial facility (105), and a single bioreactor (121) even when more than one of each of these systems is included. That is, the composite AI model (317) may be applied across many combinations of renewable energy facilities (101), industrial facilities (105), and bioreactors (121) or across individual groupings including only one of each of the renewable energy facilities (101), industrial facilities (105), and bioreactors (121) without limitation.

In accordance with one or more embodiments, the operation data (311), the set of operation parameters (313), and the set of microbe parameters (309) may be pre-processed before being processed by the composite AI model (317). Pre-processing may include activities such as, numericalization, filtering and/or smoothing of the data, scaling (e.g., normalization) of the data, feature selection, outlier removal (e.g., z-outlier filtering) and feature engineering. Feature selection includes identifying and selecting a subset of operation data with the greatest discriminative power with respect to predicting methane production (321). For example, in one embodiment, discriminative power may be quantified by calculating the strength of correlation between elements of the operation data (311) and the predicted quantities (e.g., predicted methane (321)). Consequently, in some embodiments, not all of the operation data (311) need be passed to the composite AI model. Feature engineering encompasses combining, or processing, various operation data (311) to create derived quantities. The derived quantities can be processed by the composite AI model (317). For example, the operation data (311) may be processed by one or more “basis” functions such as a polynomial basis function or a radial basis function. In some embodiments, the operation data (311) is passed to the composite AI model (317) without pre-processing. Many additional pre-processing techniques exist such that one with ordinary skill in the art would not interpret those listed here as a limitation on the present disclosure.

In accordance with one or more embodiments, the predicted methane production (321) is used to determine the set of operation parameters (313) and the set of microbe parameters (309) that optimizes methane production in the bioreactor (121). That is, the set of operation parameters (313) and the set of microbe parameters (309) define an operational state of a storage horizon configured as a bioreactor (121) for the production of methane and thus affect the output of the bioreactor (121), namely, methane production (123). Determining the optimal set of operation parameters and optimal set of microbe parameters is carried out by an optimizer (318) applied to the composite AI model (317). Upon identifying the set of operation parameters (313) and the set of microbe parameters that optimize methane production (i.e., the optimal set of operation parameters and the optimal set of microbe parameters), these sets of parameters may be applied across the bioreactor (121) and associated facilities automatically using control systems that receive commands from the bioreactor optimization system (315). The command issued from the bioreactor optimization system to the various facilities, wherein the command is to apply the new sets of optimal parameters, is indicated by Command X (323). In one or more embodiments, methane production, as monitored by at least one sensor from the plurality of sensors, and collected as underground data, is continuously monitored to ensure that the accepted optimal set of operation parameters and optimal set of microbe parameters maintain the bioreactor at optimal methane production. That is, the accepted set of operation parameters and the accepted set of microbe parameters are validated by measuring the methane production of the bioreactor and determining whether the quantity of methane is improved after adjusting the set of operation parameters (313) to the optimal set of operation parameters and adjusting the set of microbe parameters (309) to the optimal set of microbe parameters.

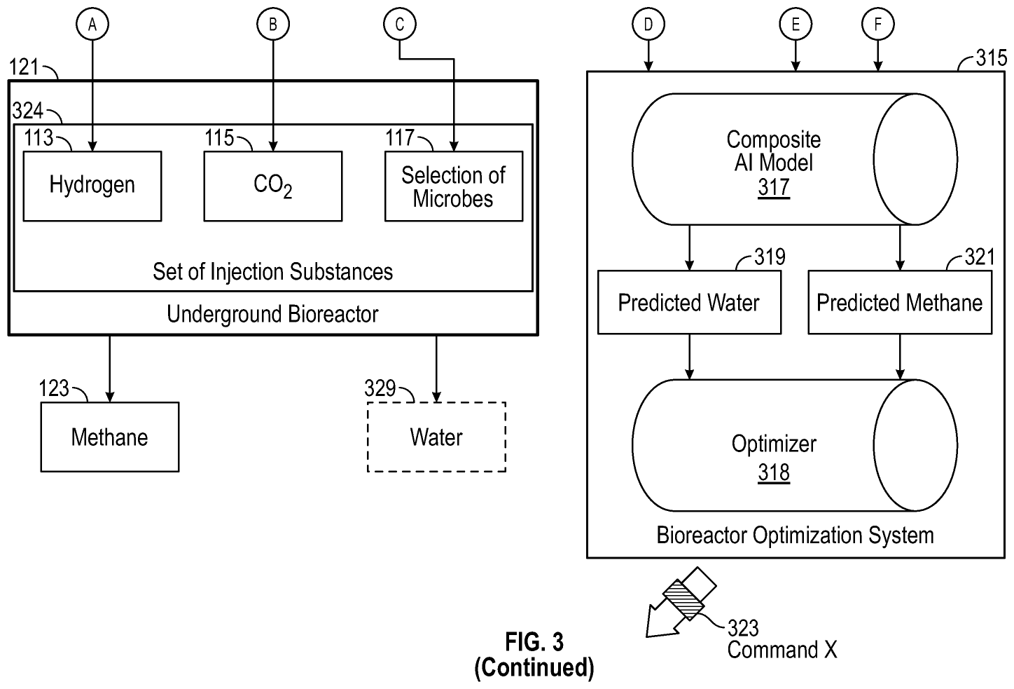

FIG. 4 depicts the use of the composite AI model (317) to predict outputs (411) including, at least, methane production (321), the composite AI model (317) and predicted methane (321) further used to determine the set of operation parameters and the set of microbe parameters that optimize the methane production, in accordance with one or more embodiments. In one or more embodiments, the composite AI model (317) is trained using previously acquired, or historic, modelling data. The modelling data is composed of observed and measured (or otherwise estimated or simulated) AI model “inputs” (401) and associated “outputs” (also known as “targets” in the context of training). The AI model inputs include the operation parameters, the operation parameters including the renewable energy facility parameters (301) and the industrial facility parameters (307), the set of microbe parameters (309), and all other data retained, including the environmental data (303) and process data (305), or derived quantities created, after pre-processing (if applied). The environmental data (303) and process data (305) may be collected over a period of time or may be acquired from analogous facilities. The outputs used for training the composite AI model, not to be confused with the outputs of the composite AI model itself (411), may include the actual amount of methane produced by the bioreactor. The AI model inputs listed are not to be considered limiting, and additional inputs may be considered in other embodiments without departing from the scope of this disclosure. For example, other optional inputs (403) may include underground data measured from inside the bioreactor, the underground data (407) including, for example, a measurement of pressure, temperature, acidity, salinity or other measurements not listed. The underground data (407) in this case may provide physical constraints on the allowable types of microbes or on the potential habitability in the bioreactor as the growth and longevity of methanogens depends, at least in part, on their environment. Another optional input (403) may be the electrolysis efficiency (405), which relates to the total amount of hydrogen produced by the renewable energy facility.

The AI model inputs (401) and other optional inputs (403) are used to train a composite AI model (317). The composite AI model (317) may be of any type known in the art. In some embodiments, multiple AI model types and/or architectures may be used to form the composite AI model (317). Generally, the AI model type and architecture with the greatest performance on a set of hold-out data is selected. Training an AI model involves processing data to develop a functional relationship between the inputs and the targets of the modelling data. The trained AI model may be described as a function relating the inputs (401) and the outputs (411). That is, the AI model may be mathematically represented as outputs=ƒ(inputs), such that given an input (401) the AI model (317) may produce an output (411). In one or more embodiments, the trained composite AI model, upon processing an input, produces two outputs, namely a predicted water production (319) and a predicted methane production (321).

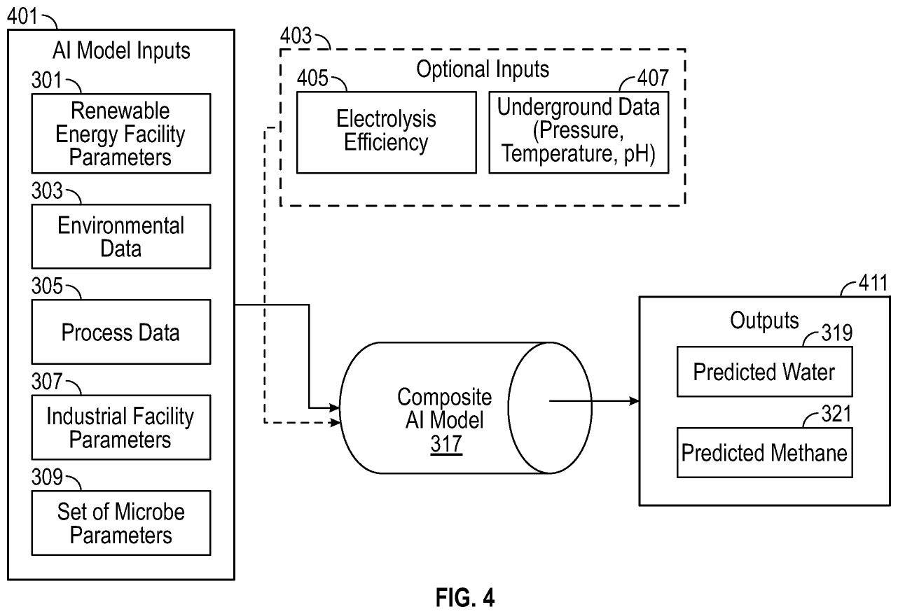

In some embodiments, the composite AI model (317) may include a plurality of AI models working in concert to predict a quantity (and/or rate) of methane produced by the bioreactor and a quantity (and/or rate) of water produced by the bioreactor. FIG. 5 depicts an example where the composite AI model (317) includes a first AI model (505), a second AI model (507), and a third AI model (515), in accordance with one or more embodiments. As shown, the first AI model (505) is used to predict a quantity (and/or rate) of hydrogen (511) produced by the renewable energy facility. Accordingly, the first AI model (505) accepts as inputs the parameters and data relevant to the production of hydrogen from the renewable energy facility, including the renewable energy facility parameters (301) and the environmental data (303), collectively referred to as first inputs (501). In some embodiments, the first AI model (505) may also accept as inputs other data relevant to the production of hydrogen (e.g., Optional First Inputs (517)), including the electrolysis efficiency from an electrolyzer used to convert electrical energy generated by the renewable energy facility into chemical energy in the form of hydrogen. Training targets for the first AI model (505) may include a measurement of the electricity and hydrogen produced by the renewable energy facility. A second AI model (507) is used to predict a quantity (and/or rate) of CO₂ (513) produced by the industrial facility. Accordingly, the second AI model (507) accepts as inputs the parameters and data relevant to the production of CO₂ from the industrial facility, including the industrial facility parameters (307) and the process data (305), collectively referred to as second inputs (503). Training targets for the second AI model may include a measurement of the CO₂ produced by the industrial facility. A third AI model (515) is used to predict a quantity (and/or rate) of methane produced by the bioreactor (321) and a quantity (and/or rate) of water produced by the bioreactor (319). The inputs for the third AI model (515) include the predicted hydrogen (511) from the first AI model (505), the predicted CO₂ (513) from the second AI model (507), and the set of microbe parameters (309) that define the selection of microbes injected into the bioreactor. In some embodiments, the third AI model (515) may accept as input other data relevant to the production of methane and water inside the bioreactor (e.g., Optional Third Inputs (521)), including underground data (407), the underground data (407) including, for example, internal measurements of the bioreactor such as pressure, temperature, acidity, and salinity. The underground data (407) may provide physical constraints on the allowable types of microbes or on the potential habitability in the bioreactor as the growth and longevity of methanogens depends, at least in part, on their environment. Training targets for the third AI model (515) may include a measurement of the methane produced by the bioreactor. Whether the composite AI model (317)) includes only one AI model, or a plurality of AI models working in concert, the output of the composite AI model (Outputs (411)) is the predicted water (319) and predicted methane (321) produced by the bioreactor.

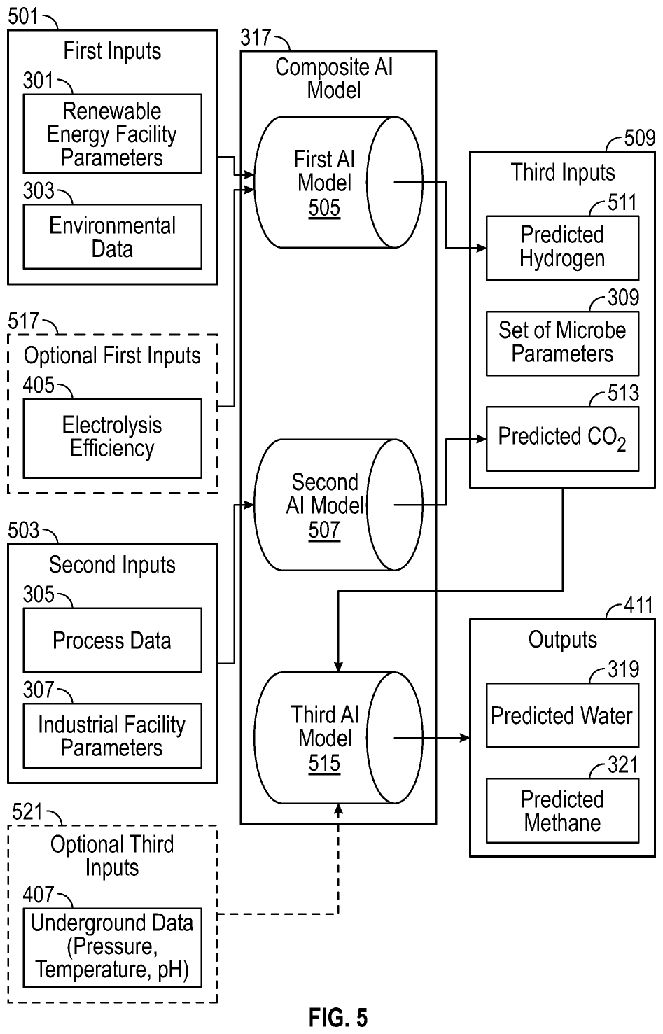

In accordance with one or more embodiments, an optimization algorithm or method is used to invert, or intelligently probe one or more inputs (e.g., set of operation parameters (313), set of microbe parameters (309)) of the composite AI model (317) to determine the set of operation parameters and the set of microbe parameters that optimize methane production given the predicted methane (321). A commonly used non-linear optimizer is the genetic algorithm (GA). An overview of the typical steps used in the genetic algorithm (GA) is provided in FIG. 6. The genetic algorithm (600) begins by generating an initial population or multiple populations (602). Here, it is emphasized that the term “population” with respect to implementations of the GA is not to be confused with the microbe population (108). A population consists of one or more “individuals.” In the context of the genetic algorithm (600), an individual is a single representation, or encoding, of the function parameters over which optimization is to occur. In the case of the storage horizon configured as a bioreactor to produce methane, the parameters over which optimization is to occur include states or values encompassed by the set of operation parameters and the set of microbe parameters. In this case, an individual is the assignment of values to each parameter while respecting any imposed constraints (e.g., not permitting the selection of microbes that cannot survive in the bioreactor based on underground data (407)). The number of individuals in a population, and the method of initially generating individuals, are hyperparameters chosen by the user. When multiple populations are used, commonly referred to as an “island” scheme, the populations need not be initialized using the same set of hyperparameters.

Once a population(s) has been generated (602), the “fitness” of every individual in the population(s) is evaluated (604). For example, in the context of the composite AI model (317) described in FIG. 5, the parameter values of an individual are used to predict a quantity (and/or rate) of produced hydrogen from the first AI model (505), predict a quantity (and/or rate) of produced CO₂ from the second AI model (507), assign a state to the selection of microbes via the set of microbe parameters, and subsequently propagate these inputs through to the third AI model (515), producing an output which is the predicted quantity (and/or rate) of methane produced by the bioreactor. Because the goal is to maximize the production of methane, individuals that result in higher outputs are considered “more fit.”

Next, a stopping criterion is checked (606). Many stopping criteria exist, including, but not limited to, the number of iterations the genetic algorithm has run, the maximum or minimum fitness score achieved by an individual, the relative change in fitness scores between iterations, and the similarity of individuals in a population, or combinations of these criteria. If the algorithm is to stop, typically, the most fit individual(s) seen during the genetic algorithm process is selected (612) and the algorithm terminates. Likewise, if the genetic algorithm continues, one or more individuals from the population(s) are selected (608). This selection may be done by simply selecting the portion of the population with the highest fitness scores, or through a tournament process, or other selection mechanism.

Once individuals have been selected (608), the individuals may be propagated through without alteration, removed, or altered through so-called crossover, mutation, and differential evolution methods to create “offspring” (610). The offspring are themselves individuals; that is, new representations, or encodings, of the function parameters. It is noted that many evolutionary methods exist to create offspring and the preceding list is not all-inclusive and should be considered non-limiting. The offspring are then evaluated for fitness (604) and the process is repeated until the genetic algorithm stopping criterion is met.

Again, the description of the genetic algorithm (GA) provided in FIG. 6 is generalized and one skilled in the art will appreciate that many modifications can be made, and are regularly made, to the genetic algorithm (GA) without departing from its intended scope. For example, an enhanced genetic algorithm (eGA) is developed by including additional features such as: enforcing deliberately diverse initial populations; using heterogenous hyperparameters for each population; dynamically updating or scheduling changes to the selection process and offspring creation process; using a unidirectional migration policy between populations; adding additional checks, such as a premature convergence check; using a self-adaptive differential evolution method; performing a localized exhaustive search in regions of stagnation or saturation.

Other non-linear optimizers may be employed to maximize the predicted quantity of methane produced and determine the optimal set of operation parameters and the optimal set of microbe parameters. The non-linear optimizer could be a Bayesian-based optimizer which elects new parameters based on an analysis of the updated posterior distribution. In this context, the level of exploration and exploitation would be determined by the user. In some embodiments, the optimal sets of parameters, as determined by the optimizer applied to the composite AI model, are validated by measuring the methane production of the bioreactor and determining whether the quantity of methane is improved after adjusting the set of operation parameters to the optimal set of operation parameters and adjusting the set of microbe parameters to the optimal set of microbe parameters.

In some embodiments, the optimizer may consider the predicted water production (319) alongside the predicted methane production (321) during the optimization. That is, the optimizer may jointly maximize methane production while minimizing water production. Though the chemical reaction for methane production, 4H₂+CO₂→2H₂O+CH₄ implies a fixed ratio of the involved quantities, the amount of water in the bioreactor capable of being extracted can be affected by the set of operation parameters and/or set of microbe parameters. Accordingly, in one or more embodiments, the optimizer may seek the set of operation parameters and the set of microbe parameters that jointly achieves the maximum methane production with the minimum water production from the storage horizon configured as the bioreactor. Mathematically, the optimization may take the form:

$${arg max}_{{S}_{1} , {S}_{2}} M + α W , \qquad ( 1 )$$

where the quantities M and W, representing the predicted methane production and the predicted water production, respectively, are determined using the trained composite AI model (317), in accordance with one or more embodiments. Further, in EQ. 1, the set of operation parameters is denoted as S₁ and the set of microbe parameters is denoted as S₂. Thus, the optimizer jointly maximizes the predicted methane production and minimizes the predicted water production over the set of operation parameters and the set of microbe parameters. EQ. 1 further makes uses of an optimization weighting factor (a). In EQ. 1, the optimization weighting factor is applied to the predicted water production, however, this need not be the case. In some embodiments, the optimization weighting factor is applied to (as a product) the predicted methane production. The optimization weighting factor serves, at least, two purposes. First, the optimization weighting factor acts to scale either the predicted methane production or the predicted water production (as shown in EQ. 1). Second, the optimization weighting factor weights either the predicted water production or the predicted methane production relative to the other output. In one or more embodiments, the optimization weighing factor is a predefined scalar such that the optimizer, when applied to a trained composite AI model processing operation data and parameterized by the set of operation parameters and the set of microbe parameters, returns a single and optimal set of operation parameters and a single and optimal set of microbe parameters. In other embodiments, the optimization weighting factor is an array such that the optimizer returns an array of optimal sets of operation parameters and an array of optimal sets of microbe parameters. In this case, the optimization weighting factor array and associated sets of operation parameters and sets of microbe parameters define a so-called Pareto front. In such a case, an individual set of operation parameters and set of microbe parameters can be selected by a subject matter expert or automatically using a predefined criterion. One with ordinary skill in the art will appreciate that maximization and minimization may be made equivalent through simple techniques such as negation. As such, the choice to represent the optimization as a maximization as shown in EQ. 1 does not limit the scope of the present disclosure. Whether done through minimization or maximization, the optimizer identifies the set (or sets) of operation parameters and the set (or sets) or microbe parameters that jointly maximize methane production and minimize water production according to the trained composite AI model.

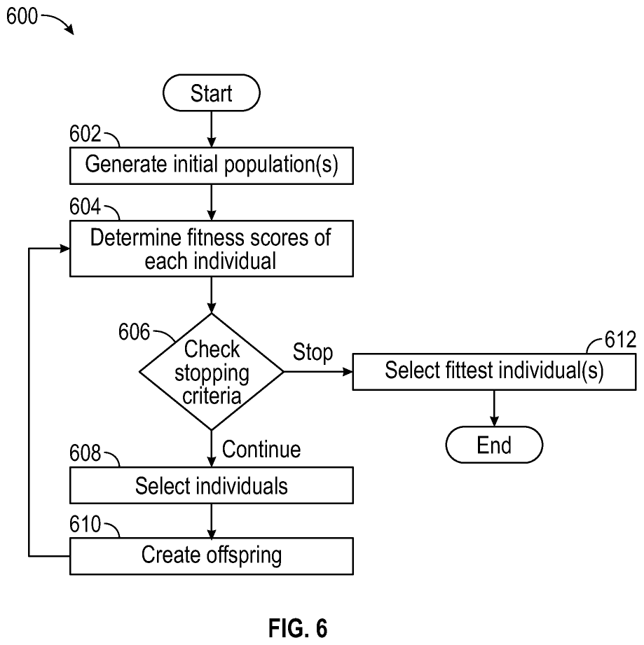

The process of evaluating process data and environmental data and determining the set of operation parameters and the set of microbe parameters that optimize methane production in an underground bioreactor that produces methane is summarized in the flow chart of FIG. 7. In Block 701, environmental data for a renewable energy facility is obtained. The environmental data may include measurements from a plurality of devices disposed throughout the renewable energy facility. The devices may include various control systems as well as sensors which measure and quantify the state of various components of the system. The measurements from the devices may include the total elapsed time of sunlight upon solar panels at a solar farm, the ambient temperature, and information from weather forecasts. Alternatively, the measurements may include the wind speed and direction at a wind farm, or other information provided by weather forecasts. In summary, the environmental data may include measurements that affect the production of energy, and therefore hydrogen, at the renewable energy facility, including measurements not listed.

In Block 703, process data from an industrial facility is obtained. The process data may include measurements from a plurality of devices disposed throughout industrial facility. The devices may include various control systems as well as sensors which measure and quantify the state of various components of the system. The measurements from the devices may include the total quantity of fossil fuels available to be consumed and thereby emit carbon dioxide, the rate of flow of various fluids to and from the industrial facility, and the temperature and/or pressure inside different chambers. The process data may further include measurements of other carbon compounds being produced by the industrial facility, such other oxocarbons like carbon monoxide and oxocarbon anions like carbonate. In summary, the process data may include measurements that affect the production of CO₂ at the industrial facility, including measurements not listed.

In one or more embodiments, the environmental data and the process data are pre-processed. Pre-processing may include numericalizing the data, scaling the data, selecting features from the data, and engineering features from the data.

In Block 705, at least some of the hydrogen produced by the renewable energy facility, at least some of the carbon dioxide produced by the industrial facility, a selection of microbes from a microbe population, the selection of microbes being defined by a set of microbe parameters, are injected into a storage horizon configured as a bioreactor that produces methane. The quantity of produced methane is controlled by, at least in part, a set of operation parameters. The set of operation parameters may include renewable energy facility parameters and industrial facility parameters. The renewable energy facility parameters define the operation of the renewable energy facility. The renewable energy facility parameters may include defining the orientation of solar panels at a solar farm, or the arrangement of wind turbines at a wind farm, or defining other characteristics that affect the generation of electricity and therefore hydrogen, or defining the amount of hydrogen injected into the bioreactor along with its characteristics, such as temperature and pressure. The industrial facility parameters define the operation of the industrial facility. The industrial facility parameters may include defining the rate at which fossil fuels are being consumed and thereby emitting CO₂, or defining the temperature and/or pressure inside different chambers, or defining the amount of CO₂ that is injected into the bioreactor along with its characteristics such as pressure or temperature. The operation parameters may also include defining properties of the injected hydrogen relative to the injected CO₂, such as a compression ratio, ratio by weight, or a relative timing of the respective injections The set of microbe parameters define the properties of the selection of microbes. The set of microbe parameters may include the quantity of injected microbes, the particular strain or combination of strains of methanogen, the ratio of the quantities belonging to each strain of methanogen in the case where more than one strain is injected, or the quantities and types of various of injected materials intended to foster and support the growth of the microbes.

In Block 707, the environmental data, the process data, the set of microbe parameters and the set of operation parameters are processed by a composite artificial intelligence (AI) model, including a first AI model, to predict methane production from an underground bioreactor. Various embodiments of the composite AI model have been described with regards to FIGS. 4 and 5. The composite AI model outputs a prediction of the quantity (or rate) of methane produced by the bioreactor, according to the set of operation parameters and a set of microbe parameters and in view of the process data and environmental data. In one or more embodiments, the composite AI model further outputs a prediction of the quantity (or rate) of water produced by the bioreactor.

In Block 709, the optimal set of operation parameters and the optimal set of microbe parameters are determined by an optimizer applied to the composite AI model such that methane production is optimized. In one or more embodiments, water production, as determined using the composite AI model, is also considered when optimizing the methane production. One possible embodiment of the optimizer, the genetic algorithm (GA) has been described above and depicted in FIG. 6. Other optimizers may be employed, such as a Bayesian-based optimizer, without limitation.

In Block 711, the set of well operation parameters and the set of microbe parameters are adjusted (e.g., through interactions between control systems in the renewable energy facility and industrial facility and a bioreactor optimization system) to their optimal values as determined using the composite AI model and optimizer. This adjustment may be performed automatically and autonomously, or may be done manually, or may be checked by a “human-in-the-loop.”

Embodiments of the present disclosure may provide at least one of the following advantages. As noted, complex interactions between the components and sub-components of a storage horizon configured as a bioreactor, above-ground installations (e.g., renewable energy facility, industrial facility, etc.), environmental conditions (e.g., weather) and subsurface conditions (e.g., pressure) exist and affect the processes of the bioreactor. Combining hydrogen output from a renewable energy facility, carbon dioxide (CO₂) from an industrial facility, and a selection of methanogenic microbes for optimal methane production is a difficult and laborious task. Further, the state and behavior of bioreactor may be transient according to the availability of reactants, and other compounds (e.g., vitamins, yeast extract, rumen fluid, etc.), for methane production which may or may not vary depending on the operation of the renewable energy facility and the industrial facility. By continuously receiving and processing environmental data and process data with a composite AI model, the bioreactor can be maintained in an optimal state greatly reducing the cost and time required to identify optimal settings which change with the transient nature of the bioreactor and the adjoining facilities. This, in turn, makes optimal use of renewable energy and restricts CO₂ from entering the environment while maximizing the production of methane.

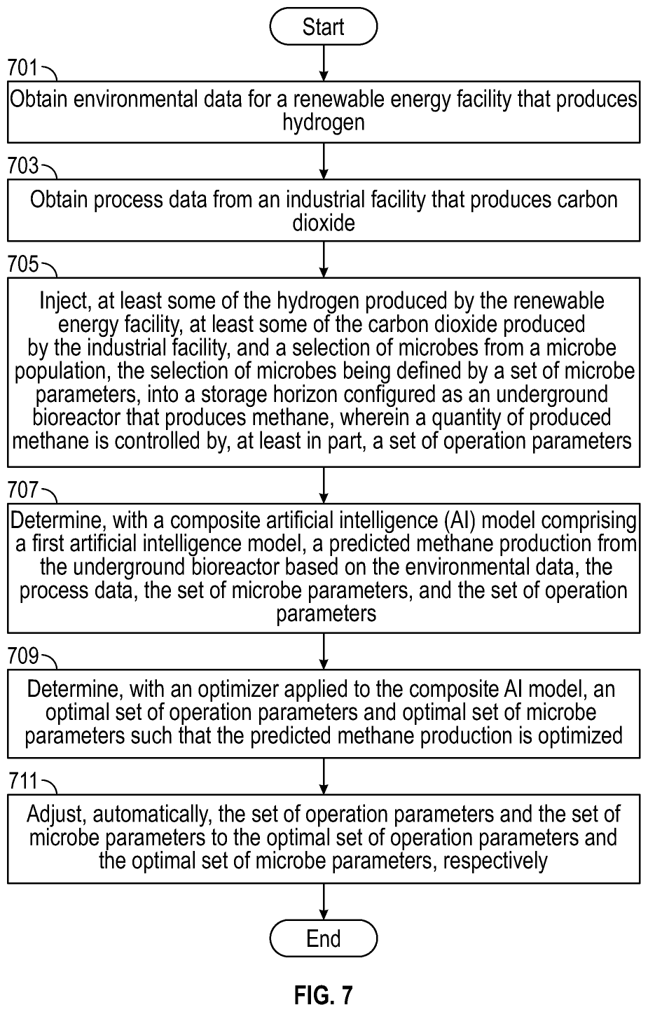

In accordance with one or more embodiments, one or more of the members of the composite AI model (317) discussed herein, such as the first AI model (505), may be a neural network. A diagram of a neural network is shown in FIG. 8. At a high level, a neural network (800) may be graphically depicted as being composed of nodes (802), where here any circle represents a node, and edges (804), shown here as directed lines. The nodes (802) may be grouped to form layers (805). FIG. 8 displays four layers (808, 810, 812, 814) of nodes (802) where the nodes (802) are grouped into columns, however, the grouping need not be as shown in FIG. 8. The edges (804) connect the nodes (802). Edges (804) may connect, or not connect, to any node(s) (802) regardless of which layer (805) the node(s) (802) is in. That is, the nodes (802) may be sparsely and residually connected. A neural network (800) will have at least two layers (805), where the first layer (808) is considered the “input layer” and the last layer (814) is the “output layer.” Any intermediate layer (810, 812) is usually described as a “hidden layer”. A neural network (800) may have zero or more hidden layers (810, 812) and a neural network (800) with at least one hidden layer (810, 812) may be described as a “deep” neural network or as a “deep learning method.” In general, a neural network (800) may have more than one node (802) in the output layer (814). In this case the neural network (800) may be referred to as a “multi-target” or “multi-output” network.

Nodes (802) and edges (804) carry additional associations. Namely, every edge is associated with a numerical value. The edge numerical values, or even the edges (804) themselves, are often referred to as “weights” or “parameters.” While training a neural network (800), numerical values are assigned to each edge (804). Additionally, every node (802) is associated with a numerical variable and an activation function. Activation functions are not limited to any functional class, but traditionally follow the form:

$$A = f ( {\sum }_{i ∈ ( incoming )} [ {( node value )}_{i} {( edge value )}_{i} ] ) , \qquad ( 3 )$$

where i is an index that spans the set of “incoming” nodes (802) and edges (804) and ƒ is a user-defined function. Incoming nodes (802) are those that, when viewed as a graph (as in FIG. 8), have directed arrows that point to the node (802) where the numerical value is being computed. Some functions for ƒ may include the linear function ƒ(x)=x, sigmoid function

$$f ( x ) = \frac{1}{1 + {e}^{- x}} ,$$

and rectified linear unit function ƒ(x)=max(0, x), however, many additional functions are commonly employed. Every node (802) in a neural network (800) may have a different associated activation function. Often, as a shorthand, activation functions are described by the function ƒ by which it is composed. That is, an activation function composed of a linear function ƒ may simply be referred to as a linear activation function without undue ambiguity.

When the neural network (800) receives an input, the input is propagated through the network according to the activation functions and incoming node (802) values and edge (804) values to compute a value for each node (802). That is, the numerical value for each node (802) may change for each received input. Occasionally, nodes (802) are assigned fixed numerical values, such as the value of 1, that are not affected by the input or altered according to edge (804) values and activation functions. Fixed nodes (802) are often referred to as “biases” or “bias nodes” (806), displayed in FIG. 8 with a dashed circle.

In some implementations, the neural network (800) may contain specialized layers (805), such as a normalization layer, or additional connection procedures, like concatenation. One skilled in the art will appreciate that these alterations do not exceed the scope of this disclosure.

As noted, the training procedure for the neural network (800) comprises assigning values to the edges (804). To begin training the edges (804) are assigned initial values. These values may be assigned randomly, assigned according to a prescribed distribution, assigned manually, or by some other assignment mechanism. Once edge (804) values have been initialized, the neural network (800) may act as a function, such that it may receive inputs and produce an output. As such, at least one input is propagated through the neural network (800) to produce an output. Recall, that a given data set will be composed of inputs and associated target(s), where the target(s) represent the “ground truth,” or the otherwise desired output. In accordance with one or more embodiments, the input of the neural network is the environmental data (which may be pre-processed), the process data (which may be pre-processed), the set of operation parameters, and set of microbe parameters and the target is methane production (given as either quantities over a pre-defined period or rates).

The neural network (800) output is compared to the associated input data target(s). The comparison of the neural network (800) output to the target(s) is typically performed by a so-called “loss function;” although other names for this comparison function such as “error function,” “misfit function,” and “cost function” are commonly employed. Many types of loss functions are available, such as the mean-squared-error function, however, the general characteristic of a loss function is that the loss function provides a numerical evaluation of the similarity between the neural network (800) output and the associated target(s). The loss function may also be constructed to impose additional constraints on the values assumed by the edges (804), for example, by adding a penalty term, which may be physics-based, or a regularization term. Generally, the goal of a training procedure is to alter the edge (804) values to promote similarity between the neural network (800) output and associated target(s) over the data set. Thus, the loss function is used to guide changes made to the edge (804) values, typically through a process called “backpropagation.”

While a full review of the backpropagation process exceeds the scope of this disclosure, a brief summary is provided. Backpropagation consists of computing the gradient of the loss function over the edge (804) values. The gradient indicates the direction of change in the edge (804) values that results in the greatest change to the loss function. Because the gradient is local to the current edge (804) values, the edge (804) values are typically updated by a “step” in the direction indicated by the gradient. The step size is often referred to as the “learning rate” and need not remain fixed during the training process. Additionally, the step size and direction may be informed by previously seen edge (804) values or previously computed gradients. Such methods for determining the step direction are usually referred to as “momentum” based methods.

Once the edge (804) values have been updated, or altered from their initial values, through a backpropagation step, the neural network (800) will likely produce different outputs. Thus, the procedure of propagating at least one input through the neural network (800), comparing the neural network (800) output with the associated target(s) with a loss function, computing the gradient of the loss function with respect to the edge (804) values, and updating the edge (804) values with a step guided by the gradient, is repeated until a termination criterion is reached. Common termination criteria are: reaching a fixed number of edge (804) updates, otherwise known as an iteration counter; a diminishing learning rate; noting no appreciable change in the loss function between iterations; reaching a specified performance metric as evaluated on the data or a separate hold-out data set. Once the termination criterion is satisfied, and the edge (804) values are no longer intended to be altered, the neural network (800) is said to be “trained”.

In accordance with one or more embodiments, the composite AI model (317), or any AI model included in the composite AI model (317) (e.g., first AI model (505)), used in the frameworks described herein may be a long short-term memory (LSTM) network. To best understand a LSTM network, it is helpful to describe the more general recurrent neural network, for which an LSTM may be considered a specific implementation.

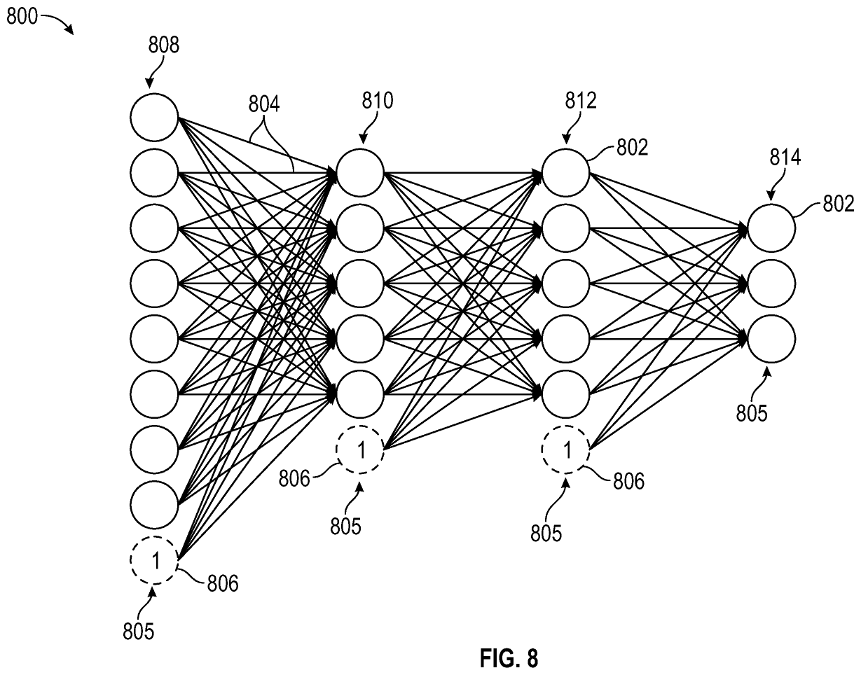

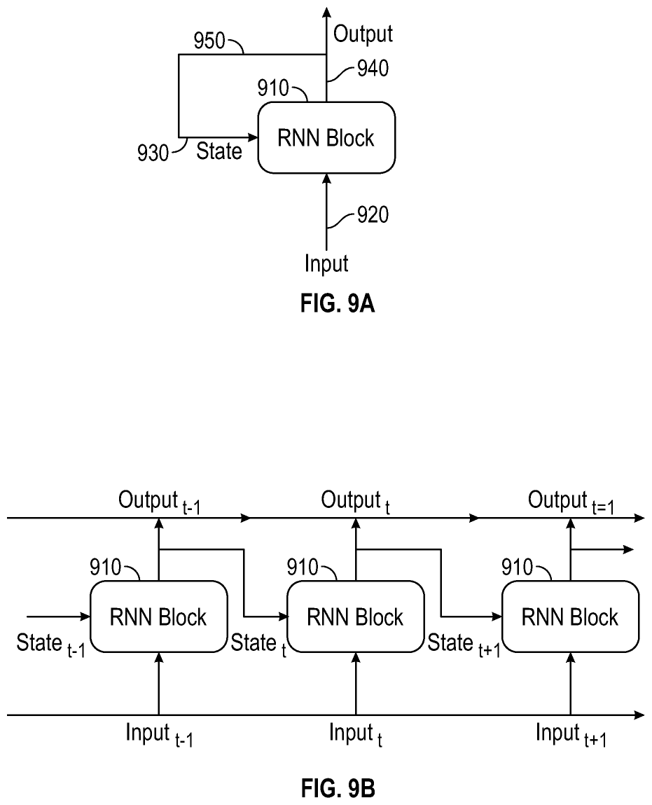

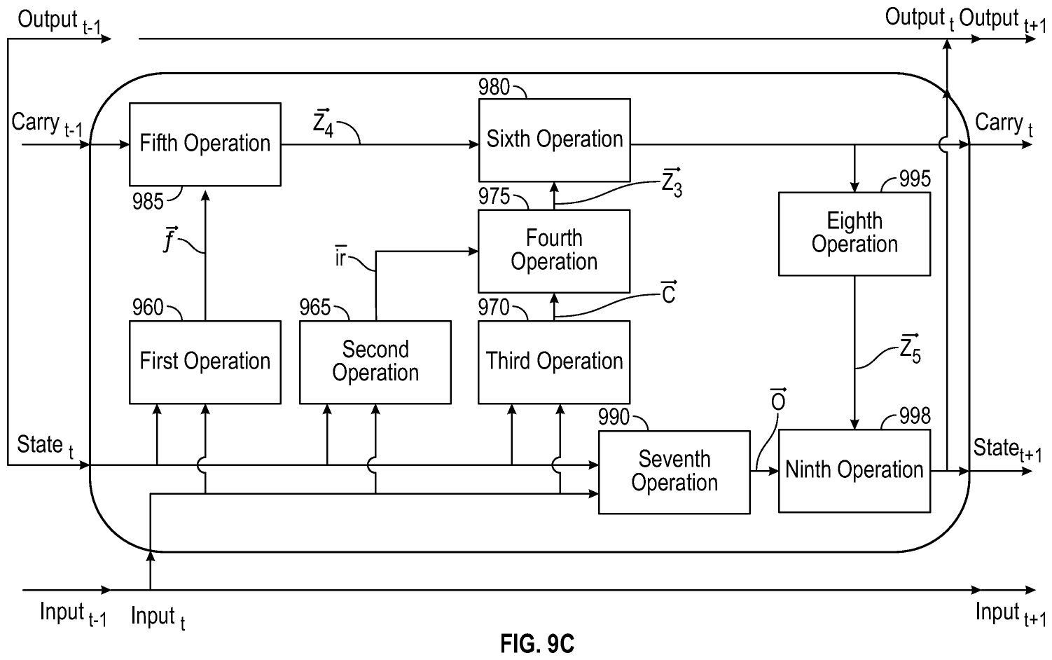

FIG. 9A depicts the general structure of a recurrent neural network (RNN). An RNN is graphically composed of an RNN Block (910) and a recurrent connection (950). The RNN Block may be thought of as a function which accepts an Input (920) and a State (930) and produces an Output (940). Without loss of generality, such a function may be written as

$$Output = RNN Block ( Input , State ) . \qquad ( 4 )$$

The RNN Block (910) generally comprises one or more matrices and one or more bias vectors. The elements of the matrices and bias vectors are commonly referred to as “weights” or “parameters” in the literature such that the matrices may be referenced as weight matrices or parameter matrices without ambiguity. It is noted that for situations with higher dimensional inputs (e.g., inputs with a tensor rank greater than or equal to 2), the weights of an RNN Block (910) may be contained in higher order tensors, rather than in matrices or vectors. For clarity, the present example will consider Inputs (920) as vectors or as scalars such that the RNN Block (910) comprises one or more weight matrices and bias vectors, however, one with ordinary skill in the art will appreciate that this choice does not impose a limitation on the present disclosure. Typically, an RNN Block (910) has two weight matrices and a single bias vector which are distinguished with an arbitrary naming nomenclature. A commonly employed naming convention is to call one weight matrix W and the other U and to reference the bias vector as \vec{b}.

An important aspect of an RNN is that it is intended to process sequential, or ordered, data; for example, a time-series. In the RNN, the Input (920) may be considered a single part of a sequence. As an illustration, consider a sequence composed of Y parts. Each part may be considered an input, indexed by t, such that the sequence may be written as sequence=[input₁, input₂, inputₜ, . . . , input_{Y-1}, input_{Y}]. Each Input (920) (e.g., input₁ of a sequence) may be a scalar, vector, matrix, or higher-order tensor. Recall that a given seismic data set is composed of N_{c} traces (or channels) and Nₜ discrete time steps. In accordance with one or more embodiments, each Input (920) (or element of a sequence) is an array of traces at a single time step. That is, each Input (920) is considered a vector with N_{c} elements.

To process a sequence, an RNN receives the first ordered Input (920) of the sequence, input₁, along with a State (930), and processes them with the RNN Block (910) according to EQ. 4 to produce an Output (940). The Output (940) may be a scalar, vector, matrix, or tensor of any rank. For the present example, the Output (940) is considered a vector with k elements. The State (930) is of the same type and size as the Output (940) (e.g., a vector with k elements). For the first ordered input, the State (930) is usually initialized with all of its elements set to the value zero. For the second ordered Input (920), input₂, of the sequence, the Input (920) is processed similarly according to EQ. 4, however, the State (930) received by the RNN Block (910) is set to the value of the Output (940) determined when processing the first ordered Input (920). This process of assigning the State (930) the value of the last produced Output (940) is depicted with the recurrent connection (950) in FIG. 9A. All the Inputs (920) in a sequence are processed by the RNN Block (910) in this manner; that is, the State (930) associated with an Input (920) is the Output (940) of the RNN Block (910) produced by the previous Input (920) (with the exception of the first Input (920) in the sequence). In some implementations, each Output (940), one for each Input (910) within a sequence, is stored for later processing and use. In other implementations, only the final Output (940), or the Output (940) which is produced when the Input (920) input_{Y} is processed by the RNN Block (910), is retained.

In greater detail, the process of the RNN Block (910), or EQ. 4, may be generally written as:

$$Output = RNN Block ( input , state ) = f ( U \cdot state + W \cdot input + \vec{b} ) , \qquad ( 5 )$$

where W, U, and \vec{b} are the weight matrices and bias vector of the RNN Block (910), respectively, and f is an “activation function.” Some functions for ƒ may include the sigmoid function

$$f ( x ) = \frac{1}{1 + {e}^{- x}} ,$$

and rectified linear unit (ReLU) function ƒ(x)=max(0, x), however, many additional functions are commonly employed.

To further illustrate a RNN, a pseudo-code implementation of a RNN is as follows.

RNN Algorithm
Note:
N_{c} = input length
k = output length
W ϵ [symbol]
U ϵ [symbol]
\vec{b} ϵ [symbol]
1: state = [0₁, 0₂, ... , 0ₖ₋₁, 0ₖ]^{T}
2: for input in sequence:
3: \vec{z}₁ = matmul(U, state)
4: \vec{z}₂ = matmul(W, input)
5: output = ƒ(\vec{z}₁ + \vec{z}₂ + \vec{b})
6: state = output

In keeping with the previous examples, both the inputs and the outputs are considered vectors of lengths N_{c} and k, respectively, however, in general, this need not be the case. With the lengths of these vectors defined, the shapes of the weight matrices, bias vector, and State (930) vector may be specified. To begin processing a sequence, the State (930) vector is initialized with values of zero as shown in line 1 of the pseudo-code. Note that in some implementations, the number of inputs contained within a sequence may not be known or may vary between sequences. One with ordinary skill in the art will recognize that an RNN may be implemented without knowing, beforehand, the length of the sequence to be processed. This is demonstrated in line 2 of the pseudo-code by indicating that each input in the sequence will be processed sequentially without specifying the number of inputs in the sequence. Once an Input (920) is received, a matrix multiplication operator is applied between the weight matrix U and the State (930) vector. The resulting product is assigned to the temporary variable \vec{z}₁. Likewise, a matrix multiplication operator is applied between the weight matrix W and the Input (910) with the result assigned to the variable \vec{z}₂. For the present example, due the Input (920) and Output (940) each being defined as vectors, the products in lines 3 and 4 of the pseudo-code may be expressed as matrix multiplications, however, in general, the dot product between the weight matrix and corresponding State (930) or Input (920) may be applied. The Output (940) is determined by summing \vec{z}₁, \vec{z}₂, and the bias vector \vec{b} and applying the activation function ƒ elementwise. The State (930) is set to the Output (940) and the whole process is repeated until each Input (920) in a sequence has been processed.

FIG. 9B depicts an “unrolled” version of the RNN of FIG. 9A. Unrolling the RNN allows one to see how the sequential inputs, indexed by t, produce sequential outputs and how the state is passed through various inputs of the sequence. It is noted that while the “unrolled” depiction shows multiple RNN Blocks (910), these blocks are the same such that they are comprised of the same weight matrices and bias vector.

As previously stated, generally, training a machine-learned model requires that pairs of inputs and one or more targets (i.e., a training dataset) are passed to the machine-learned model. During this process the machine-learned model “learns” a representative model which maps the received inputs to the associated outputs. In the context of an RNN, the RNN receives a sequence, wherein the sequence can be partitioned into one or more sequential parts (Inputs (920) above), and maps the sequence to an overall output, which may also be a sequence. To remove ambiguity and distinguish the overall output of an RNN from any intermediate Outputs (940) produced by the RNN Block (910), the overall output will be referred to herein as a RNN result. In other words, an RNN receives a sequence and returns a RNN result. The training procedure for a RNN comprises assigning values to the weight matrices and bias vector of the RNN Block (910). For brevity, the elements of the weight matrices and bias vector will be collectively referred to as the RNN weights. To begin training the RNN weights are assigned initial values. These values may be assigned randomly, assigned according to a prescribed distribution, assigned manually, or by some other assignment mechanism. Once the RNN weights have been initialized, the RNN may act as a function, such that it may receive a sequence and produce a RNN result. As such, at least one sequence may be propagated through the RNN to produce a RNN result. For training, a training dataset is composed of one or more sequences and desired RNN results, where the desired RNN results represent the “ground truth”, or the true RNN results that should be returned for the given sequences. For clarity, and consistency with previous discussions of machine-learned model training, the desired or true RNN results will be referred to as targets. When processing sequences, the RNN result produced by the RNN is compared to the associated target. The comparison of a RNN result to the target(s) is typically performed by a loss function. As before, other names for this comparison function such as “error function” and “cost function” are commonly employed. Many types of loss functions are available, such as the mean squared error function, however, the general characteristic of a loss function is that the loss function provides a numerical evaluation of the similarity between the RNN result and the associated target(s). The loss function may also be constructed to impose additional constraints on the values assumed by RNN weights, for example, by adding a penalty term, which may be physics-based, or a regularization term. Generally, the goal of a training procedure is to alter the RNN weights to promote similarity between the RNN results and associated targets over the training dataset. Thus, the loss function is used to guide changes made to the RNN weights, typically through a process called “backpropagation through time.”

A long short-term memory (LSTM) network may be considered a specific, and more complex, instance of a recurrent neural network (RNN). FIG. 9C is an unrolled depiction of a LSTM where the internal components of the LSTM are displayed as labelled abstractions. A LSTM, like a RNN, has a recurrent connection, such that the output produced by a single input in a sequence is forwarded as the state to be used with the subsequent input. However, an LSTM also possesses another “state-like” data structure commonly referred to as the “carry.” The carry, like the state and input may be a scalar, vector, matrix, or tensor of any rank depending on the context of the application. Like unto the description of the RNN, for simplicity, the carry will be considered a vector in the following discussion of the LSTM. The LSTM receives an input, state, and carry and produces an output and a new carry. The output and the new carry are passed to the LSTM as the state and carry for the subsequent input. This sequential process, indexed by t, may be described functionally as:

$$( {output}_{t} , {carry}_{t} ) = LSTM Block ( {input}_{t} , {carry}_{t - 1} , {state}_{t} ) = LSTM Block ( {input}_{t} , {carry}_{t - 1} , {output}_{t - 1} ) , \qquad ( 6 )$$

where the LSTM Block, like the RNN Block, comprises one or more weight matrices and bias vectors and the processing steps necessary to transform an input, state, and carry to an output and new carry.

LSTMs may be configured in a variety of ways, however, the processes depicted in FIG. 9C are the most common. As shown in FIG. 9C, an LSTM Block receives an input (inputₜ), a state (stateₜ), and a carry (carryₜ₋₁). Again, assuming that the inputs, carry, and outputs are all vectors, the weights of the LSTM Block may be considered to reside in eight matrices and four bias vectors. These matrices and vectors are conventionally named Wᵢ, Uᵢ, W_{ƒ}, U_{ƒ}, W_{c}, U_{c}, Wₒ, Uₒ and \vec{b}ᵢ, \vec{b}_{ƒ}, \vec{b}_{c}, \vec{b}ₒ, respectively. The processes of the LSTM Block are as follows. Block 960 represents the following first operation:

$$\vec{f} = {a}_{1} ( {U}_{f} \cdot {state}_{t} + {W}_{f} \cdot {input}_{t} + {\vec{b}}_{f} ) , \qquad ( 7 )$$

where a₁ is an activation function applied elementwise to the result of the parenthetical expression and the resulting vector is \vec{ƒ}. Block 965 implements the following second operation:

$$\vec{i} = {a}_{2} ( {U}_{i} \cdot {state}_{t} + {W}_{i} \cdot {input}_{t} + {\vec{b}}_{i} ) , \qquad ( 8 )$$

Where a₂ is an activation function which may be the same or different to a₁ and is applied elementwise to the result of the parenthetical expression. The resulting vector is \vec{t}. Block 970 implements the following third operation:

$$\vec{c} = {a}_{3} ( {U}_{c} \cdot {state}_{t} + {W}_{c} \cdot {input}_{t} + {\vec{b}}_{c} ) , \qquad ( 9 )$$

where a₃ is an activation function which may be the same or different to either a₁ or a₂ and is applied elementwise to the result of the parenthetical expression. The resulting vector is \vec{c}. In block 975, vectors \vec{i} and \vec{c} are multiplied according to a fourth operation:

$${\vec{z}}_{3} = \vec{i} ⊙ \vec{c} , \qquad ( 10 )$$

where ⊙ (indicates the Hadamard product (i.e., elementwise multiplication). Likewise, in block 985 the carry vector from the previous sequential input (carryₜ₋₁) vector and the vector \vec{ƒ} are multiplied according to a fifth operation:

$${\vec{z}}_{4} = {carry}_{t - 1} ⊙ \vec{f} . \qquad ( 11 )$$

The results of the operations of blocks 975 and 985 (\vec{z}₃ and \vec{z}₄, respectively) are added together in block 980, a sixth operation, to form the new carry (carryₜ):

$${carry}_{t} = {\vec{z}}_{3} + {\vec{z}}_{4} . \qquad ( 12 )$$

In block 990, the current input and state vectors are processed according to a seventh operation:

$$\vec{o} = {a}_{4} ( {U}_{o} \cdot {state}_{t} + {W}_{o} \cdot {input}_{t} + {\vec{b}}_{o} ) , \qquad ( 13 )$$

Where a₄ is an activation function which may be unique or identical to any other used activation function and is applied elementwise to the result of the parenthetical expression. The result is the vector \vec{o}. In block 995, an eighth operation, the new carry (carryₜ) is passed through an activation function a₅. The activation a₅ is usually the hyperbolic tangent function but may be any known activation function. The eighth operations (block 995) may be represented as:

$${\vec{z}}_{5} = {a}_{5} ( {carry}_{t} ) . \qquad ( 14 )$$

Finally, the output of the LSTM Block (outputₜ) is determined in block 998 by taking the Hadamard product of \vec{z}₅ and \vec{o}, a ninth operation shown mathematically as:

$${output}_{t} = {\vec{z}}_{5} ⊙ \vec{o} . \qquad ( 15 )$$

The output of the LSTM Block is used as the state vector for the subsequent input. Again, as in the case of the RNN, the outputs of the LSTM Block applied to a sequence of inputs may be stored and further processed or, in some implementations, only the final output is retained. While the processes of the LSTM Block described above used vector inputs and outputs, it is emphasized that an LSTM network may be applied to sequences of any dimensionality. In these circumstances the rank and size of the weight tensors will change accordingly. One with ordinary skill in the art will recognize that there are many alterations and variations that can be made to the general LSTM structure described herein, such that the description provided does not impose a limitation on the present disclosure.

While multiple embodiments using different AI models have been suggested, one skilled in the art will appreciate that this process, of determining the set of operation parameters and set of microbe parameters that optimize methane production, is not limited to the listed AI models. AI models such as a random forest, support vector machines, or non-parametric methods such as K-nearest neighbors may be readily inserted into this framework and do not depart from the scope of this disclosure.

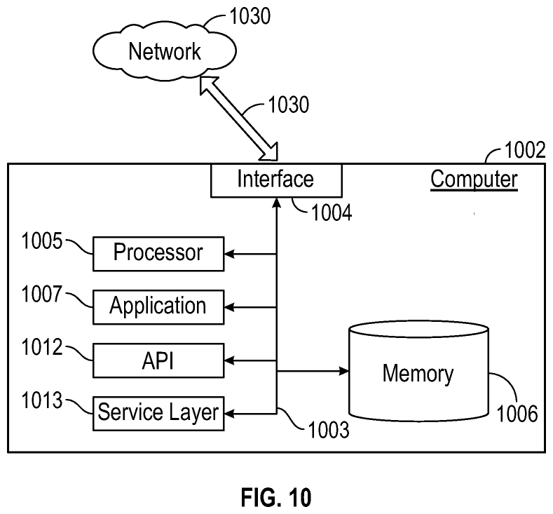

Embodiments may be implemented on a computer system. FIG. 10 is a block diagram of a computer system (1002) used to provide computational functionalities associated with described algorithms, methods, functions, processes, flows, and procedures as described in the instant disclosure, according to one or more embodiments. The illustrated computer (1002) is intended to encompass any computing device such as a server, desktop computer, laptop/notebook computer, wireless data port, smart phone, personal data assistant (PDA), tablet computing device, one or more processors within these devices, or any other suitable processing device such as an edge computing device, including both physical or virtual instances (or both) of the computing device. An edge computing device is a dedicated computing device that is, typically, physically adjacent to the process or control with which it interacts. For example, the AI model may be implemented on an edge computing device in order to quickly provide optimal sets of operation parameters and microbe parameters to associated devices or their controllers (e.g., control system).

Additionally, the computer (1002) may include a computer that includes an input device, such as a keypad, keyboard, touch screen, or other device that can accept user information, and an output device that conveys information associated with the operation of the computer (1002), including digital data, visual, or audio information (or a combination of information), or a GUI.

The computer (1002) can serve in a role as a client, network component, a server, a database or other persistency, or any other component (or a combination of roles) of a computer system for performing the subject matter described in the instant disclosure. In some implementations, one or more components of the computer (1002) may be configured to operate within environments, including cloud-computing-based, local, global, or other environment (or a combination of environments).

At a high level, the computer (1002) is an electronic computing device operable to receive, transmit, process, store, or manage data and information associated with the described subject matter. According to some implementations, the computer (1002) may also include or be communicably coupled with an application server, e-mail server, web server, caching server, streaming data server, business intelligence (BI) server, or other server (or a combination of servers).

The computer (1002) can receive requests over network (1030) from a client application (for example, executing on another computer (1002) and responding to the received requests by processing the said requests in an appropriate software application. In addition, requests may also be sent to the computer (1002) from internal users (for example, from a command console or by other appropriate access method), external or third-parties, other automated applications, as well as any other appropriate entities, individuals, systems, or computers.

Each of the components of the computer (1002) can communicate using a system bus (1003). In some implementations, any or all of the components of the computer (1002), both hardware or software (or a combination of hardware and software), may interface with each other or the interface (1004) (or a combination of both) over the system bus (1003) using an application programming interface (API) (1012) or a service layer (1013) (or a combination of the API (1012) and service layer (1013). The API (1012) may include specifications for routines, data structures, and object classes. The API (1012) may be either computer-language independent or dependent and refer to a complete interface, a single function, or even a set of APIs. The service layer (1013) provides software services to the computer (1002) or other components (whether or not illustrated) that are communicably coupled to the computer (1002). The functionality of the computer (1002) may be accessible for all service consumers using this service layer. Software services, such as those provided by the service layer (1013), provide reusable, defined business functionalities through a defined interface. For example, the interface may be software written in JAVA, C++, or other suitable language providing data in extensible markup language (XML) format or another suitable format. While illustrated as an integrated component of the computer (1002), alternative implementations may illustrate the API (1012) or the service layer (1013) as stand-alone components in relation to other components of the computer (1002) or other components (whether or not illustrated) that are communicably coupled to the computer (1002). Moreover, any or all parts of the API (1012) or the service layer (1013) may be implemented as child or sub-modules of another software module, enterprise application, or hardware module without departing from the scope of this disclosure.

The computer (1002) includes an interface (1004). Although illustrated as a single interface (1004) in FIG. 10, two or more interfaces (1004) may be used according to particular needs, desires, or particular implementations of the computer (1002). The interface (1004) is used by the computer (1002) for communicating with other systems in a distributed environment that are connected to the network (1030). Generally, the interface (1004) includes logic encoded in software or hardware (or a combination of software and hardware) and operable to communicate with the network (1030). More specifically, the interface (1004) may include software supporting one or more communication protocols associated with communications such that the network (1030) or interface's hardware is operable to communicate physical signals within and outside of the illustrated computer (1002).

The computer (1002) includes at least one computer processor (1005). Although illustrated as a single computer processor (1005) in FIG. 10, two or more processors may be used according to particular needs, desires, or particular implementations of the computer (1002). Generally, the computer processor (1005) executes instructions and manipulates data to perform the operations of the computer (1002) and any algorithms, methods, functions, processes, flows, and procedures as described in the instant disclosure.

The computer (1002) also includes a memory (1006) that holds data for the computer (1002) or other components (or a combination of both) that can be connected to the network (1030). The memory may be a non-transitory computer readable medium. For example, memory (1006) can be a database storing data consistent with this disclosure. Although illustrated as a single memory (1006) in FIG. 10, two or more memories may be used according to particular needs, desires, or particular implementations of the computer (1002) and the described functionality. While memory (1006) is illustrated as an integral component of the computer (1002), in alternative implementations, memory (1006) can be external to the computer (1002).

The application (1007) is an algorithmic software engine providing functionality according to particular needs, desires, or particular implementations of the computer (1002), particularly with respect to functionality described in this disclosure. For example, application (1007) can serve as one or more components, modules, applications, etc. Further, although illustrated as a single application (1007), the application (1007) may be implemented as multiple applications (1007) on the computer (1002). In addition, although illustrated as integral to the computer (1002), in alternative implementations, the application (1007) can be external to the computer (1002).

There may be any number of computers (1002) associated with, or external to, a computer system containing computer (1002), wherein each computer (1002) communicates over network (1030). Further, the term “client,” “user,” and other appropriate terminology may be used interchangeably as appropriate without departing from the scope of this disclosure. Moreover, this disclosure contemplates that many users may use one computer (1002), or that one user may use multiple computers (1002).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A method, comprising:
obtaining environmental data for a renewable energy facility that produces hydrogen;
obtaining process data from an industrial facility that produces carbon dioxide;
injecting, at least some of the hydrogen produced by the renewable energy facility, at least some of the carbon dioxide produced by the industrial facility, and a selection of microbes from a microbe population, the selection of microbes being defined by a set of microbe parameters, into a storage horizon configured as an underground bioreactor that produces methane, wherein a quantity of produced methane is controlled by, at least in part, a set of operation parameters;
determining, with a composite artificial intelligence (AI) model comprising a first artificial intelligence model, a predicted methane production from the underground bioreactor based on the environmental data, the process data, the set of microbe parameters, and the set of operation parameters;
determining, with an optimizer applied to the composite AI model, an optimal set of operation parameters and optimal set of microbe parameters such that the predicted methane production is optimized; and
adjusting, automatically, the set of operation parameters and the set of microbe parameters to the optimal set of operation parameters and the optimal set of microbe parameters, respectively.

2. The method of claim 1, further comprising:
determining, with the composite AI model, a predicted water production from the underground bioreactor,
wherein determining, with the optimizer, the optimal set of operation parameters and the optimal set of microbe parameters comprises jointly maximizing the predicted methane production while minimizing the predicted water production.

3. The method of claim 1 further comprising:
measuring a quantity of methane produced by the bioreactor; and
validating the optimal set of operation parameters and the optimal set of microbe parameters by determining whether the quantity of methane is improved after adjusting the set of operation parameters to the optimal set of operation parameters and adjusting the set of microbe parameters to the optimal set of microbe parameters.

4. The method of claim 1, wherein the optimizer is a genetic algorithm.

5. The method of claim 1, wherein the hydrogen production comprises electrolysis using electricity generated by the renewable energy facility.

6. The method of claim 1, wherein the set of operation parameters comprises:
a set of renewable energy facility parameters that control the operation of the renewable energy facility; and
a set of industrial facility parameters that control the operation of the industrial facility.

7. The method of claim 1, wherein the set of operation parameters comprises a compression ratio of the hydrogen and carbon dioxide injected into the storage horizon.

8. The method of claim 1, wherein the set of microbe parameters comprise a strain of the injected selection of microbes.

9. The method of claim 1, further comprising obtaining underground data from at least one sensor disposed within the bioreactor, wherein the composite AI model is informed by the underground data.

10. The method of claim 1, wherein the composite AI model further comprises:
a second AI model that determines a predicted hydrogen quantity from the renewable energy facility based on the environmental data and renewable energy facility parameters; and
a third AI model that determines a predicted carbon dioxide quantity from the industrial facility based the process data and industrial facility parameters;
wherein the first AI model determines the methane production based on the predicted hydrogen quantity, the predicted carbon dioxide quantity, and the set of microbe parameters.

11. The method of claim 10, wherein the first AI model is a long short-term memory network.

12. A system, comprising:
a renewable energy facility that produces hydrogen and an industrial facility that produces carbon dioxide, wherein the operation of the renewable energy facility and the industrial facility is defined by a set of operation parameters;
a plurality of facility devices disposed throughout the renewable energy facility and the industrial facility, the plurality of facility devices gathering operation data from the renewable energy facility and the industrial facility;
a microbe population providing a selection of microbes, wherein properties of the selection of microbes are defined by a set of microbe parameters;
a storage horizon configured as an underground bioreactor that produces methane, wherein the quantity of produced methane is controlled, at least in part, by a set of injection materials, the injection materials comprising:
the hydrogen from the renewable energy facility,
the carbon dioxide from the industrial facility, and
the selection of microbes from the microbe population;
a control system configured to adjust one or more facility devices in the plurality of facilities; and
a computer configured to:
obtain the operation data for the renewable energy facility and the industrial facility,
obtain the set of operation parameters for the renewable energy facility and the industrial facility,
obtain the set of microbe parameters for the selection of microbes,
determine, with a composite AI model comprising a first AI model, a predicted methane production based on the operation data, the set of operation parameters, and the set of microbe parameters,
determine, with an optimizer applied to the composite AI model, an optimal set of operation parameters and optimal set of microbe parameters such that the predicted methane production is optimized, and
adjust, automatically, the set of operation parameters and the set of microbe parameters to the optimal set of operation parameters and the optimal set of microbe parameters, respectively, to optimize the predicted methane production.

13. The system of claim 12:
wherein the operation data comprises:
environmental data from the renewable energy facility, and
process data from the industrial facility,
wherein the set of operation parameters comprises:
a set of renewable energy facility parameters, and
a set of industrial facility parameters

14. The system of claim 12, further comprising at least one sensor disposed within the underground bioreactor gathering underground data.

15. The system of claim 12, wherein the computer is further configured to:
determine, with the composite AI model, a predicted water production from the underground bioreactor,
wherein determining, with the optimizer, the optimal set of operation parameters and the optimal set of microbe parameters comprises jointly maximizing the predicted methane production while minimizing the predicted water production.

16. The system of claim 12, wherein the computer is further configured to:
measure a quantity of methane produced by the bioreactor; and
validate the optimal set of operation parameters and set of microbe parameters by determining whether the quantity of methane is improved after adjusting the set of operation parameters to the optimal set of operation parameters and adjusting the set of microbe parameters to the optimal set of microbe parameters, respectively.

17. The system of claim 12, wherein the first AI model is a long short-term memory network.

18. The system of claim 13, wherein the composite AI model further comprises:
a second AI model that determines a predicted hydrogen quantity from the renewable energy facility based on the environmental data and renewable energy facility parameters; and
a third AI model that determines a predicted carbon dioxide quantity from the industrial facility based the process data and industrial facility parameters;
wherein the first AI model determines the methane production based on the predicted hydrogen quantity, the predicted carbon dioxide quantity, and the set of microbe parameters.

19. The system of claim 12, wherein the hydrogen production comprises electrolysis using electricity generated by the renewable energy facility.

20. A non-transitory computer-readable memory comprising computer-executable instructions stored thereon that, when executed on a processor, cause the processor to perform steps comprising:
obtaining environmental data for a renewable energy facility that produces hydrogen;
obtaining process data from an industrial facility that produces carbon dioxide;
determining, with a composite artificial intelligence (AI) model, a predicted methane production from an underground bioreactor based on the environmental data, the process data, and a set of microbe parameters in view of a set of operation parameters that control the operation of the renewable energy facility and the industrial facility;
determining, with an optimizer applied to the composite AI model, an optimal set of operation parameters and optimal set of microbe parameters such that the predicted methane production is optimized; and
adjusting, automatically, the set of operation parameters and the set of microbe parameters to the optimal set of operation parameters and the optimal set of microbe parameters, respectively.
